(19) United States
(12) Patent Application Publication
(10) Pub. No.: US 2024/0167062 A1
(43) Pub. Date: May 23, 2024

(54) HYDROGEL SYSTEM FOR CONTROLLING OXYGEN CONCENTRATION

(71) Applicant: Ramot at Tel-Aviv University Ltd., Tel-Aviv (IL)

(72) Inventors: Iftach YACOBY, Tel-Aviv (IL); Itzhak GRINBERG, Tel-Aviv (IL); Oren BENZVI, Tel-Aviv (IL); Ehud GAZIT, Tel-Aviv (IL); Lihi ADLER-ABRAMOVICH, Tel-Aviv (IL)

(73) Assignee: Ramot at Tel-Aviv University Ltd., Tel-Aviv (IL)

(21) Appl. No.: 18/281,982

(22) PCT Filed: Mar. 16, 2022
(86) PCT No.: PCT/IL2022/050299

§ 371 Date: Sep. 14, 2023

Related U.S. Application Data
(60) Provisional application No. 63/161,589, filed on Mar. 16, 2021.
(60) Provisional application No. 63/161,603, filed on Mar. 16, 2021.

(51) Int. Cl. C12P 3/00; A61K 9/06; A61K 38/05; C01B 3/02; C01C 1/02

(52) U.S. Cl. CPC C12P 3/00; A61K 9/06; A61K 38/05; C01B 3/02; C01C 1/02

(57) ABSTRACT

Methods which utilize a self-assembled structure formed of a plurality of peptides being of from 2 to 6 amino acids in length, and comprising at least one aromatic amino acid residue, wherein the self-assembled structure is capable of interacting with oxygen, are provided herewith. The methods may be for controlling (e.g., reducing) a concentration of free oxygen in an environment, and/or for transporting oxygen from a first environment to a second environment. Further described herein is a self-assembled structure as described herein having oxygen interacted therewith. Further described herein is a composition comprising the self-assembled structure and substance which is oxygen-sensitive and/or participates in an oxygen-sensitive reaction incorporated in the self-assembled structure, systems comprising such a composition and methods employing the composition or system. Further described herein are articles-of-manufacturing comprising any of the self-assembled structures or compositions as described herein and an oxygen-sensitive substance and uses thereof.

### RELATED APPLICATION/S

This application claims the benefit of priority under 35 USC § 119(e) of U.S. Provisional Patent Application Nos. 63/161,589 and 63/161,603 co-filed on Mar. 16, 2021, the contents of each of which are incorporated herein by reference in their entirety.

### FIELD AND BACKGROUND OF THE INVENTION

The present invention, in some embodiments thereof, relates to materials, and more particularly, but not exclusively, to self-assembled structures usable in controlling oxygen concentrations, for example, reducing an oxygen concentration in an environment, transporting oxygen, and/or protecting substances against oxygen damage, to systems and articles-of-manufacturing comprising same and to methods utilizing same.

Oxygen (O₂) is an important compound, in view of its essential role in aerobic metabolism, as well as its prominent role in destructive processes such as corrosion. However, its gaseous nature and poor solubility in liquids such as water limit the degree to which oxygen concentrations can be controlled.

The interaction of oxygen with proteins in biological systems usually occurs through metal co-factors or flavins. Oxygen scavenging in industrial applications may utilize oxidation of iron powder, hydrocarbons, ascorbic acid, photosensitive dyes, unsaturated fatty acids, or immobilized yeast [Lopez-Rubio et al., Food Rev Int 2004, 20:357-387]. Porous sachets containing iron powder and sodium chloride (as a catalyst of iron oxidation) are commonly used to decrease oxygen levels in packaged products, sometimes further including activated carbon, which adsorbs various gases.

Perfluorocarbons exhibit relatively high solubility of gases, including oxygen, which has led to applications such as artificial blood products, liquid breathing (e.g., via liquid in lungs), delivery of oxygen to wounds to accelerate healing, and tissue preservation.

Self-assembled peptide-based hydrogels are environmentally friendly, easily synthesized, soft, elastic, and biocompatible materials which mainly consist of aqueous content [Fichman & Gazit, Acta Biomaterialia 2014, 10:1671-1682; Fleming & Ulijn, Chem Soc Rev 2014, 43:8150-8177]. Supramolecular self-assembly serves as a key approach for the formation of such bulk hydrogels. Specifically, low molecular weight hydrogelators have been widely explored, particularly for biotechnological and medical applications [Fichman & Gazit, Acta Biomaterialia 2014, 10:1671-1682; Fleming & Ulijn, Chem Soc Rev 2014, 43:8150-8177].

The self-assembled ultra-short peptide building blocks are easy to fabricate, inexpensive and can be simply chemically and biologically decorated [Mahler et al., Adv Mater 2006, 18:1365-1370; Jayawarna et al., Adv Mater 2006, 18:611-614]. These advantages are unique to peptide hydrogels as compared to many natural or synthetic hydrogels [Fichman & Gazit, Acta Biomaterialia 2014, 10:1671-1682].

A notable example of peptide-based hydrogels is the Fluorenylmethyloxycarbonyl-diphenylalanine (Fmoc-FF) aromatic dipeptide building block that can self-assemble in aqueous solutions to form nano-scaled ordered fibrils that form a 3D hydrogel of remarkable mechanical rigidity [Mahler et al., Adv Mater 2006, 18:1365-1370; Jayawarna et al., Adv Mater 2006, 18:611-614; Hauser & Zhang, Chem Soc Rev 2010, 39:2780-2790]. It was demonstrated that by taking advantage of π-π interactions between aromatic rings, self-assembly of short peptide molecules can easily be achieved [Dudkovic & Zukoski, Langmuir 2014, 30:4493-4500; Orbach et al., Biomacromolecules 2009, 10:2646-2651; Orbach et al., Biomacromolecules 2012, 28:2015-2022; Adler-Abramovich & Gazit, Chem Soc Rev 2014, 43:6881-6893].

International Patent Application No. PCT/IL2014/050208 (published as WO 2014/132262), which is incorporated by reference as if fully set forth herein, describes physically discrete hydrogel particles, which each comprise a three-dimensional network made of self-assembled peptides comprising at least one aromatic amino acid and an aqueous medium.

Many enzymatic reactions are hindered by oxygen damage, e.g., by reaction reversal as for nitroreductases, or by irreversible deactivation as for hydrogenases and nitrogenases. Such reactions require a strictly anaerobic environment to be effective, thus limiting their practical and applicative use.

Fossil fuels still constitute over 80% of the total primary energy consumed by humanity worldwide, despite depleting reserves and major environmental drawbacks. Furthermore, current methods of energy production from renewable sources, lack the means to efficiently store and transport energy from the time and place of production to when and where it is needed.

The [FeFe]-hydrogenase (HydA) enzyme, which can be efficiently purified from the green algae Chlamydomonas reinhardtii [Yacoby et al., PLoS One 2012, 7:e35886], can be used to produce hydrogen gas. H₂ is considered one of the best energy carriers for storing energy and releasing it in high yields. Its efficient production and utilization in fuel cells can be catalyzed by HydA and has raised much interest [Plumere et al., Nat Chem 2014, 6:822-827]. However, HydA is extremely sensitive to oxygen, which irreversibly inactivates it. Therefore, H₂ production by HydA requires a strictly anaerobic environment, limiting its practicality [Ghirardi et al., Chem Soc Rev 2009, 38:52-61].

Direct injection of electrons into the enzymes has been suggested as an attractive approach for driving the reaction and storing renewable energy [Schlager et al., J Mater Chem A 2017, 5:2429-2443].

Enzyme immobilization and encapsulation have been reported to enhance enzymatic production and stabilize the enzymes [Obert & Dave, J Am Chem Soc 1999, 121:12192-12193].

Additional background art includes International Patent Application Nos. PCT/IL2006/001174 (published as WO 2007/043048), PCT/IL2011/000435 (published as WO 2011/151832), PCT/IL2018/050773 (published as WO 2019/012545), and PCT/IL2019/050788, Adler-Abramovich & Gazit [Chem Soc Rev 2014, 43:6881-6893]; Dudukovic & Zukoski [Langmuir 2014, 30:4493-4500]; Fleming & Ulijn [Chem Soc Rev 2014, 43:8150-8177]; Hauser & Zhang [Chem Soc Rev 2010, 39:2780-2790]; Jayawarna et al. [Adv Mater 2006, 18:611-614]; Jayawarna et al. [Acta Biomater 2009, 5:934-943]; Orbach et al. [Biomacromolecules 2009, 10:2646-2651]; Orbach et al. [Biomacromolecules 2012, 28:2015-2022]; Panda et al. [ACS Appl Mater Interfaces 2010, 2:2839-2848]; RoseFigura et al. [Biochemistry 2011, 50: 1556-1566]; Schnaider et al. [Nano Lett 2020, 20:1590-1597]; Smith et al. [Adv Mater 2008, 20:37-41]; Ulijn & Smith [Chem Soc Rev 2008, 37:664-675]; Widboom et al. [Nature 2007, 447:342-345]; Yang et al. [J Mater Chem 2007, 17:850-854]; and Zhang [Interface Focus 2017, 7:20170028]; all being incorporated by reference as if fully set forth herein.

### SUMMARY OF THE INVENTION

According to an aspect of some embodiments of the present invention there is provided a method of controlling a concentration of free oxygen in an environment, the method comprising contacting the environment with a self-assembled structure formed of a plurality of peptides each being of 2 to 6 amino acid residues in length, wherein in each of the peptides, at least one of the amino acid residues is an aromatic amino acid residue, and wherein the self-assembled structure is capable of interacting with oxygen, thereby controlling the concentration of free oxygen.

According to some of any of the embodiments described herein, the self-assembled structure is a fibrillar structure (e.g., comprises a fibril).

According to some of any of the embodiments described herein, the structure comprises a nanostructure (e.g., a fibrillar nanostructure).

According to some of any of the embodiments described herein, the self-assembled structure forms a 3-dimensional network.

According to some of any of the embodiments described herein, the self-assembled structure is in a form of a hydrogel, including discrete hydrogel particles as described herein in any of the respective embodiments.

According to some of any of the embodiments described herein, contacting the environment with a plurality of discrete hydrogel particles each comprising the self-assembled structure.

According to some of any of the embodiments described herein, at least one, or each, peptide in the plurality of peptides comprises an end-capping moiety.

According to some of any of the embodiments described herein, the end-capping moiety is aromatic.

According to some of any of the embodiments described herein, the end-capping moiety is Fmoc.

According to some of any of the embodiments described herein, the end-capping moiety is attached to the N-terminus of the peptide.

According to some of any of the embodiments described herein, at least one, or each, of the aromatic amino acids is phenylalanine.

According to some of any of the embodiments described herein, the peptide is a dipeptide, and in some embodiments it is a homodipeptide.

According to some of any of the embodiments described herein, the dipeptide comprises diphenylalanine.

According to some of any of the embodiments described herein, the dipeptide is Fmoc-diphenylalanine.

According to some of any of the embodiments described herein, the self-assembled structure is capable of absorbing oxygen in an amount of at least 0.02 mg oxygen per mg of the self-assembled structure.

According to some of any of the embodiments described herein, the self-assembled structure is capable of interacting with oxygen such that the oxygen is adjacent to an aromatic ring of each of at least three aromatic amino acid residues.

According to some of any of the embodiments described herein, the environment is a fluid environment (e.g., comprises a liquid and/or a gas).

According to some of any of the embodiments described herein, the environment is an animate substrate.

According to some of any of the embodiments described herein, controlling the concentration of free oxygen is reducing a concentration of the free oxygen in the environment.

According to some of any of the embodiments described herein, controlling the concentration of free oxygen is in an environment external to the self-assembled structure.

According to some of any of the embodiments described herein, controlling the concentration of free oxygen is in an internal environment of the self-assembled structure.

According to some of any of the embodiments described herein, the self-assembled structure further comprises an oxygen-sensitive substance incorporated therein, the method being for protecting (e.g., maintaining an activity) of the oxygen-sensitive substance.

According to some of any of the embodiments described herein, the self-assembled structure further comprises a substance which participates in an oxygen-sensitive reaction incorporated therein, the method being for performing the reaction under aerobic conditions.

According to an aspect of some embodiments of the present invention there is provided a method of transporting oxygen from a first environment to a second environment, the method comprising: contacting the first environment with a self-assembled structure as defined herein in any of the respective embodiments and any combination thereof, to thereby obtain the self-assembled structure having oxygen interacted therewith; and contacting the second environment with the self-assembled structure having oxygen interacted therewith, thereby releasing oxygen from the self-assembled structure into the second environment.

According to some of any of the embodiments described herein, the method further comprises contacting the first environment with a self-assembled structure obtained by the releasing oxygen from the self-assembled structure having oxygen interacted therewith.

According to some of any of the embodiments described herein, the second environment is an oxygen-deficient environment.

According to some of any of the embodiments described herein, the first environment has a higher oxygen concentration than the second environment.

According to some of any of the embodiments described herein, the second environment is characterized by a continuous depletion of oxygen.

According to some of any of the embodiments described herein, the continuous depletion of oxygen is associated with metabolism of a living organism.

According to some of any of the embodiments described herein, the second environment comprises a tissue.

According to some of any of the embodiments described herein, the method is for enhancing healing of tissue damage.

According to some of any of the embodiments described herein, the method is for inhibiting growth of an anaerobic organism in the second environment.

According to some of any of the embodiments described herein, the method is for enhancing aerobic metabolism in the second environment.

According to some of any of the embodiments described herein, the aerobic metabolism is associated with degradation of a waste product.

According to some of any of the embodiments described herein, the second environment comprises an aqueous environment.

According to some of any of the embodiments described herein, the first environment is characterized by an oxygen partial pressure of at least 0.15 atmospheres.

According to some of any of the embodiments described herein, the self-assembled structure is dispersed in a liquid.

According to some of any of the embodiments described herein, the liquid is an aqueous liquid.

According to some of any of the embodiments described herein, the liquid comprises fibers and/or gel particles comprising the self-assembled structure.

According to some of any of the embodiments described herein, the method further comprises effecting flow of the liquid from the first environment to the second environment.

According to an aspect of some embodiments of the present invention there is provided a self-assembled structure as defined herein in any of the respective embodiments and any combination thereof having oxygen interacted therewith.

According to some of any of the embodiments described herein, an amount of oxygen interacted therewith is at least 0.02 mg oxygen per mg of the structure.

According to an aspect of some embodiments of the present invention there is provided a composition comprising a substance which is oxygen-sensitive and/or which participates in an oxygen-sensitive reaction, the substance being incorporated in a self-assembled structure as defined herein in any of the respective embodiments and any combination thereof.

According to some of any of the embodiments described herein, the substance is an enzyme.

According to some of any of the embodiments described herein, the enzyme is such that catalyzes a redox reaction.

According to some of any of the embodiments described herein, the enzyme is selected from the group consisting of a hydrogenase, a nitroreductase, and a nitrogenase.

According to some of any of the embodiments described herein, the hydrogenase comprises an [FeFe]-hydrogenase.

According to some of any of the embodiments described herein, the [FeFe]-hydrogenase is a Chlamydomonas reinhardtii [FeFe]-hydrogenase.

According to some of any of the embodiments described herein, the composition further comprises an electron transfer mediator.

According to some of any of the embodiments described herein, the composition is in electrical contact with an electrical power source.

According to an aspect of some embodiments of the present invention there is provided a system comprising the composition as described herein in any of the respective embodiments and any combination thereof, and a current collector configured for being attached to an electrical power source.

According to some of any of the embodiments described herein, the system further comprises a reservoir configured for collecting a gas and/or liquid formed by the substance.

According to some of any of the embodiments described herein, the substance comprises a hydrogenase and the gas comprises H₂.

According to some of any of the embodiments described herein, the H₂ is for use as a fuel in a fuel cell.

According to some of any of the embodiments described herein, the substance comprises a nitrogenase and the gas and/or liquid comprises ammonia and/or a salt thereof.

According to an aspect of some embodiments of the present invention there is provided a method of reducing or oxidizing a substrate, the method comprising contacting the substrate with the composition as described herein in any of the respective embodiments and any combination thereof, and applying electrical power to the composition, thereby reducing or oxidizing the substrate.

According to some of any of the embodiments described herein, the substance comprises a hydrogenase, the method being for producing H₂.

According to some of any of the embodiments described herein, the method further comprises using the H₂ as a fuel in a fuel cell.

According to some of any of the embodiments described herein, the substrate comprises water.

According to some of any of the embodiments described herein, the substance comprises a nitrogenase, the method being for producing ammonia and/or a salt thereof.

According to some of any of the embodiments described herein, the substrate comprises N₂.

According to some of any of the embodiments described herein, the method is effected in the presence of O₂ at a partial pressure of at least 0.002 atmosphere, optionally at least 0.02 atmosphere.

According to an aspect of some embodiments of the present invention there is provided an article-of-manufacturing comprising a substance which is oxygen-sensitive incorporated in a self-assembled structure as defined herein in any of the respective embodiments and any combination thereof.

According to an aspect of some embodiments of the present invention there is provided an article-of-manufacturing comprising a self-assembled structure as defined herein in any of the respective embodiments and any combination thereof and an oxygen-sensitive substance.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof.

Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents an image of an inverted vial containing an exemplary self-supporting hydrogel (near the top of the image) formed from FmocFF end-capped dipeptide.

FIG. 2 presents a scanning electron microscopy (SEM) image of an exemplary FmocFF hydrogel.

FIG. 3 presents a tunneling electron microscopy (TEM) image of nanofibrils in an exemplary FmocFF hydrogel.

FIGS. 4A-C present graphs showing storage modulus (G′) and loss modulus (G″) (in units of Pa) as a function of time upon formation of FmocFF hydrogel, as determined by in situ time sweep oscillation measurements (FIGS. 4A and 4B) or upon application and release of shear stress in a cyclic manner (FIG. 4C).

FIG. 5 presents images of vials containing methyl viologen in solution or in an exemplary FmocFF gel, upon exposure to air for 0, 20, 60 or 120 minutes (bleaching of color indicates oxidation of methyl viologen by O₂).

FIGS. 6A-B present an image of specialized FireSting™ probe vials used to determine O₂ penetration into exemplary FmocFF hydrogels versus aqueous solutions (FIG. 6A) and a graph showing O₂ concentration in buffer, BocFF fibrils, cross-linked alginate, Fmoc-F5-Phe, agarose and FmocFF hydrogels as a function of time of exposure to O₂ (initiated by uncapping of anaerobic vials; FIG. 6B), as determined using an optical meter system as shown in FIG. 6A (n≥4).

FIGS. 7A-7D present bar graphs showing gas emission rates for N₂ (FIG. 7A), argon (FIG. 7B), CO₂ (FIG. 7C) and O₂ (FIG. 7D) from air-saturated FmocFF gel, agarose gel and aqueous solution, as determined by membrane inlet mass spectrometry (n≥7; * indicates p<0.05, ** indicates p<0.01, *** indicates p<0.001; error bars represent standard error).

FIG. 8 presents a graph showing O₂ concentration in double distilled water (DDW) and agarose and FmocFF hydrogels (prepared with DDW) as a function of time of exposure to O₂ (initiated by uncapping of anaerobic vials), as determined using an optical meter system (n≥4).

FIG. 9 presents a bar graph showing the rate of O₂ penetration into double distilled water (DDW) and agarose and FmocFF hydrogels (prepared with DDW), as determined from the linear phase of the data presented in FIG. 8.

FIGS. 10A-10D present bar graphs showing gas emission rates for N₂ (FIG. 10A), argon (FIG. 10B), CO₂ (FIG. 10C) and O₂ (FIG. 10D) from air-saturated FmocFF and agarose gels (prepared with DDW) and solution in DDW, as determined by membrane inlet mass spectrometry (n=7; * indicates p<0.05, ** indicates p<0.01, *** indicates p<0.001; error bars represent standard error).

FIGS. 11A-11C present molecular graphics images of a simulation system and modeled FmocFF fibril, with a simulated FmocFF fibril segment in a 100 Å³ water box, and O₂ molecules shown as pairs of attached spheres (FIG. 11A), a side view of the simulated FmocFF fibril segment with scale bar showing thickness (FIG. 11B), and a top-down view of the simulated FmocFF fibril segment with scale bars showing length and width (FIG. 11C).

FIG. 12 presents a bar graph showing the percentage of O₂, Ar, and CO₂ molecules bound to the surface (solid bars) and interior (striped bars) of an FmocFF fibril, as determined by molecular dynamics simulation (error bars represent standard error).

FIG. 13 presents a distribution plots of residence time (ns) per binding event for Ar, CO₂, and O₂ binding to an FmocFF fibril, as determined by molecular dynamics simulation.

FIGS. 14A and 14B present schematic depictions of interactions between the FmocFF fibril and O₂ in the most prevalent binding mode of O₂, binding mode I (FIG. 14A), and in the second most prevalent binding mode of O₂, binding mode II (FIG. 14B); O₂ depicted as pair of spheres, interacting moieties of FmocFF are shown with thick lines, differences in shading indicate peptide to which each interacting moiety belongs to.

FIG. 15 presents a schematic depiction of H₂ production according to some embodiments of the invention, whereby the O₂-sensitive enzyme HydA1 is encapsulated in an FmocFF hydrogel, FmocFF fibrils bind O₂ during the exposure period, preventing interaction of O₂ with HydA1 and maintaining its enzymatic activity; the HydA1 is activated by sodium dithionite (DT) and methyl viologen (MV) as electron donor and mediator, respectively, and the produced H₂ diffuses out of the hydrogel.

FIG. 16 is a bar graph showing specific activity of [FeFe]-hydrogenase (HydA) in H₂ production (in units of μmole H₂ per mg HydA per minute) under anaerobic conditions (no O₂) or upon exposure to 2.1% O₂, in an exemplary FmocFF hydrogel or in an aqueous solution (H₂ production determined by gas chromatography analysis).

FIG. 17 presents images of vials containing a hydrogel (right vial) or aqueous solution (left vial) comprising methyl viologen; methyl viologen exhibited color (bottom of right vial) in hydrogel, indicating low oxygen level, whereas solution was colorless within 5 minutes of exposure to O₂, indicating oxidation by O₂.

FIG. 18 presents a graph showing H₂ accumulation in vial headspace adjacent to a hydrogel (unbroken line) or aqueous solution (dashed line) exposed to O₂, as a function of time following addition of sodium dithionite and methyl viologen.

FIG. 19 presents an image of inverted vials containing exemplary hydrogels comprising 5 mg/ml (left vial), 10 mg/ml (center vial), and 20 mg/ml (right vial) FmocFF.

FIGS. 20A-B present a bar graph showing residual activity of HydA in aqueous solution and in FmocFF hydrogels comprising 5, 10 or 20 mg/ml FmocFF, following a 20 minute exposure to air (residual activity calculated as percentage of the activity measured in gels unexposed to air) (FIG. 20A), and a graph showing residual activity of HydA1 in FmocFF hydrogels (5 mg/ml) as a function of time of exposure to atmospheric O₂, and an exponential fit (and associated R² value) to the data points (error bars represent standard error) (FIG. 20B).

FIGS. 21A-B are bar graphs showing H₂ production by HydA in solution or in agarose or FmocFF hydrogels under anaerobic conditions and after 20 minutes exposure to air, with activity determined by H₂ production rate in units of μmole H₂ per mg HydA per minute (FIG. 21A) and as residual activity after 20 minutes exposure to air as percentage of the activity in gels unexposed to air (FIG. 21B).

### DESCRIPTION OF SPECIFIC EMBODIMENTS OF THE INVENTION

The present invention, in some embodiments thereof, relates to materials, and more particularly, but not exclusively, to self-assembled structures (e.g., hydrogels) usable in controlling oxygen concentrations, for example, reducing an oxygen concentration in an environment, transporting oxygen, and/or protecting substances against oxygen damage, to systems and articles-of-manufacturing comprising same and to methods utilizing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that fibrillar structures (e.g., nanofibrils) made of self-assembled aromatic peptides (e.g., FmocFF) may interact with oxygen (e.g., presumably via hydrophobic interactions, by trapping oxygen molecules within binding pockets within the structure). This feature facilitates trapping of oxygen while embedded within a solid matrix, for example, gels formed by the aromatic peptide fibrils themselves and/or other gel-forming agents; as well as oxygen transport, while the fibrils are suspended within a solvent (which is compatible with fibril integrity).

Embodiments of the present invention relate to methods utilizing the interaction between such fibrillar structures and oxygen for controlling oxygen concentration, for example, reducing oxygen concentration is a fluid substance or environment and/or transporting oxygen.

Examples for applications of these features include, without limitation, oxygen transport from anaerobic hoods, gloveboxes and clean rooms; oxygen transport, from food, drugs, cosmetic products and alcoholic beverage preservation packages and devices, such as refrigerators and containers; as preservatives for machines which continuously recycle liquids, such as cooling electronic devices (e.g., computers), combustion engine radiators, washing machines, and dishwashers: medical applications, such as circulating baths for speeding up wound and burn healing; and inhibition of anaerobic organisms (e.g., pathogenic bacteria) and/or anoxic conditions (e.g., in water).

The present inventors have utilized the oxygen control capability of such hydrogels for devising a novel methodology for protecting oxygen-sensitive substances and/or reactions from molecular oxygen damage and greatly increasing O₂ tolerance of participants in such reactions, such as enzymatic reactions. This is achieved through encapsulation of an oxygen-sensitive substance and/or a substance participating in such a reaction (e.g., an oxygen-sensitive enzyme) in self-assembled peptide-based hydrogel.

While reducing the present invention to practice, the inventors have shown that such hydrogel encapsulation can allow reactants to diffuse in and products to diffuse out, while the deleterious effects of O₂ are reduced, and the reaction remains active for longer periods of time, in comparison to a corresponding reaction in a solution. For example, exemplary hydrogenase-containing hydrogels exhibited activity for several hours in the presence of an O-containing atmosphere which was comparable to the activity exhibited by hydrogenase-containing solutions for only about 5 minutes in the presence of the same atmosphere.

The present invention can be useful, for example, in converting electricity (e.g., produced from renewable sources) into usable chemical fuels, by utilizing catalysts such as enzymes (e.g., due to the natural ability of enzymes to catalyze chemical reactions in high yield, scalable, cost efficient manner, and under mild conditions). Examples of commercially important reactions catalyzed by oxygen-sensitive enzymes—which may be protected using some embodiments of the invention—include, without limitation, the formation of H₂ (a potentially important fuel) by hydrogenases under reducing conditions, and nitrogen fixation by nitrogenases (e.g., formation of ammonia from N₂).

Embodiments of the present invention relate to compositions, systems and methods utilizing a self-assembled structure having incorporated therein an oxygen-sensitive substance or a substance participating in an oxygen-sensitive reaction in oxygen-sensitive processes such as, for example, enzymatically-catalyzed processes, rubber manufacturing processes, and energy conversion processes. Embodiments of the present invention further relate to products and articles-of-manufacturing comprising a self-assembled structure having incorporated therein an oxygen-sensitive substance.

Self-Assembled Structures:

The self-assembled structure according to the present embodiments is formed of a plurality of peptides each being independently of 2, 3, 4, 5 or 6, or of 2, 3 or 4, or of 2, amino acid residues in length. In each of the peptides, one or more of the amino acid residues is an aromatic amino acid residue, as defined herein. The peptides in the plurality of peptides that form the self-assembled structure can be the same or different, and when different, the peptides may differ from one another by number and/or type of the amino acid residues.

According to some of any of the embodiments described herein, at least 50%, or at least 60%, or at least 70%, or at least 80% or at least 90%, or at least 95%, or at least 98%, or all, of the peptides, are the same peptides in terms of number and type of amino acid residues.

According to some of any of the embodiments described herein, the self-assembled structure is a fibrillar structure (e.g., comprises one or more fibrils). In some embodiments, the structures are self-assembled upon forming aromatic interactions between the aromatic portion of the aromatic molecules that form the structures.

Herein, the term “fibril”, and its adjectival form “fibrillar”, refers to a structure characterized by a narrow cross-section, preferably having an average diameter of less than 10% (optionally less than 1%) of the length of the structure (along a long axis thereof).

According to some of any of the embodiments described herein, the structure comprises a nanostructure (e.g., a fibrillar nanostructure).

As used herein the phrase “nanostructure” refers to a structure having a diameter or a cross-section in at least one dimension thereof of less than 1 m (preferably less than about 100 nm, more preferably less than about 50 nm, and even more preferably less than about 20 nm, e.g., of about 10 nm).

As used herein the phrase “fibrillar nanostructure” refers to a filament or fiber having a diameter or a cross-section width of less than 1 m (preferably less than about 100 nm, more preferably less than about 50 nm, and even more preferably less than about 20 nm, e.g., of about 10 nm). The length of the fibrillar nanostructure (along its long axis) is preferably at least 1 nm, more preferably at least 10 nm, even more preferably at least 100 nm and even more preferably at least 500 nm.

According to some of any of the embodiments described herein, the self-assembled structure forms a 3-dimensional network.

According to some of any of the embodiments described herein, the self-assembled structure is in a form of a hydrogel, for example, a hydrogel comprising an aqueous liquid in addition to the self-assembled structure.

As used herein and in the art, the term “hydrogel” refers to a gel, typically semi-solid, material that comprises 3-dimensional fibrous networks formed of natural or synthetic chains, typically containing more than 80%, or more than 90% or more than 95% or more than 99%, by weight or by volume, water or an aqueous solution.

As used herein the phrase “fibrous network” refers to a set of connections formed between a plurality of fibrous components. Herein, the fibrous components are optionally composed of a plurality of fibrils (e.g., fibrillar nanostructures), at least a portion of which, or each, being formed upon self-assembly of aromatic building blocks (e.g., aromatic amino acid).

According to some of any of the embodiments described herein, the aqueous solution is water.

According to some of any of the embodiments described herein, the aqueous solution is a buffer.

According to some of any of the embodiments described herein, the self-assembled structure (e.g., hydrogel) is formed upon contacting the plurality of peptides with an aqueous solution (e.g., water or buffer) under conditions that allow or promote the self-assembly, as previously described. Exemplary conditions are described in the Examples section that follows.

In some of any of the embodiments relating to a hydrogel, the self-assembled structure is in a form a plurality of discrete hydrogel particles, each comprising the self-assembled structure; for example, hydrogel particles as described in International Patent Application Publication WO 2014/132262, the contents of which are incorporated herein by reference, especially the descriptions therein of hydrogel particles. The hydrogel particles may optionally be dispersed in a liquid (e.g., aqueous liquid), which may be substantially the same and/or different than a liquid (e.g., aqueous liquid) comprised by the hydrogel particles.

As described herein, the peptides forming the self-assembled structure according to embodiments of the invention include at least one aromatic amino acid residue. According to some of any of the embodiments described herein, in at least a portion, or in all of the plurality of peptides, each of the amino acid residues in the peptide is an aromatic amino acid residue.

By “aromatic amino acid” it is meant an amino acid, or an amino acid residue in a peptide comprising same, that has an aromatic moiety or group, as defined herein, is its side chain. In exemplary embodiments, an aromatic amino acid has, for example, a substituted or unsubstituted naphthalenyl or a substituted or unsubstituted phenyl, in its side chain. The substituted phenyl may be, for example, pentafluorophenyl, iodophenyl, biphenyl and/or nitrophenyl.

As used herein, the phrase “aromatic group” or “aromatic moiety” describes a monocyclic or polycyclic moiety having a completely conjugated pi-electron system. The aromatic group can be an all-carbon moiety or can include one or more heteroatoms such as, for example, nitrogen, sulfur or oxygen. The aromatic group can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine.

Exemplary aromatic groups include, for example, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic groups that can serve as the side chain within the aromatic amino acid described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl. The aromatic group can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

In some of any of the embodiments described herein, the aromatic molecule comprises at least one aromatic moiety that is an all-carbon aromatic moiety, e.g., an aryl as defined herein.

In some of any of the embodiments described herein, the aromatic amino acid is phenylalanine. In some such embodiments, at least a portion, or each peptide in the structure comprises a plurality of phenylalanine residues.

In some of any of the embodiments described herein, the peptide is a dipeptide (i.e., having two amino acid residues), and in some embodiments it is a homodipeptide (i.e., having two amino acid residues which are identical with respect to their side-chain). The dipeptide (e.g., homodipeptide) may optionally be modified by an end-capping moiety according to any of the respective embodiments described herein).

Exemplary aromatic homodipeptides include, but are not limited to, phenylalanine-phenylalanine dipeptide (diphenylalanine peptide), naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide (e.g., (pentafluoro-phenylalanine)-(pentafluoro-phenylalanine) dipeptide), (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide (e.g., (4-fluoro-phenylalanine)-(4-fluoro-phenylalanine) dipeptide), (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide, each of which dipeptides may optionally be modified (e.g., according to any of the respective embodiments described herein). In some of any of embodiments described herein relating to a homodipeptide, the homodipeptide is phenylalanine-phenylalanine and/or naphthylalanine-naphthylalanine dipeptide.

Representative examples of suitable aromatic peptides other than homodipeptides include, without limitation, phenylalanine-glycine (Phe-Gly), phenylalanine-arginine-glycine-aspartic acid (Phe-Arg-Gly-Asp), and arginine-glycine-aspartic acid-phenylalanine (Arg-Gly-Asp-Phe) peptides, each of which peptides may optionally be modified (e.g., according to any of the respective embodiments described herein).

According to some of any of the embodiments described herein, at least a portion, or each peptide in the plurality of peptides (according to any of the respective embodiments described herein) comprises an end-capping moiety.

The phrase “end-capping moiety”, as used herein, refers to a moiety that when attached to the terminus of a peptide, modifies the peptide terminus. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”). Fmoc is an exemplary end-capping moiety.

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively, the —COOH group of the C-terminus end-capping may be modified to an amide group.

Other end-capping modifications include replacement of the amine and/or carboxyl with a different moiety, such as hydroxyl, thiol, halide, alkyl, aryl, alkoxy, aryloxy and the like, as these terms are defined herein.

In some embodiments of the present invention, a self-assembled structure is made of a plurality of peptides and all of the peptides composing the structure are end-capping modified. In some of these embodiments, the aromatic amino acids are modified only at the N-terminus or the C-terminus thereof, e.g., resulting in a structure that has a negative net charge or a positive net charge, respectively. In another embodiment, the aromatic amino acids are modified at both the N-terminus and the C-terminus thereof, e.g., resulting in an uncharged structure.

According to some of any of the embodiments described herein, the peptide is end-capping modified at the N-terminus thereof.

The end-capping moiety may optionally be aromatic or non-aromatic. According to some of any of the respective embodiments described herein, the end capping moiety is an aromatic end-capping moiety.

According to some of any of the embodiments described herein, the end-capping moiety is attached to the N-terminus of the peptide, and the end-capping moiety is an aromatic moiety (e.g., Fmoc).

Representative examples of aromatic end capping moieties suitable for N-terminus modification include, without limitation, fluorenylmethyloxycarbonyl (Fmoc). Representative examples of aromatic end capping moieties suitable for C-terminus modification include, without limitation, benzyl, benzyloxycarbonyl (Cbz), trityl and substituted trityl groups.

Representative examples of non-aromatic end capping moieties suitable for N-terminus modification include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

According to some embodiments, the end-capping modified peptides comprise dipeptides, and according to some embodiments, are homodipeptides (e.g., according to any of the respective embodiments described herein). In some such embodiments, the dipeptides are aromatic dipeptides, in which at least one, preferably both, of the amino acid residues is an aromatic amino acid residue.

Representative examples of such end-capping modified homodipeptides include, without limitation, end-capping modified phenylalanine-phenylalanine (Phe-Phe) dipeptides, end-capping modified naphthylalanine-naphthylalanine (Nal-Nal) dipeptides, end-capping modified (pentafluoro-phenylalanine)-(pentafluoro-phenylalanine) dipeptides, end-capping modified (iodo-phenylalanine)-(iodo-phenylalanine), end-capping modified (4-phenyl phenylalanine)-(4-phenyl phenylalanine) and end-capping modified (p-nitro-phenylalanine)-(p-nitro-phenylalanine). Fmoc-diphenylalanine (i.e., diphenylalanine which Fmoc attached to the N-terminus thereof) is an exemplary dipeptide which comprises phenylalanine and an end-capping moiety.

Contemplated are homodipeptides, and more preferably aromatic homodipeptides in which each of the amino acids comprises an aromatic moiety, such as, but not limited to, substituted or unsubstituted naphthalenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine When substituted, the phenyl, naphthalenyl or any other aromatic moiety includes one or more substituents such as, but not limited to, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

In some of any of these embodiments, the end-capping modified peptide is an N-terminus modified peptide.

In some of any of these embodiments, the end-capping modified peptide comprises an aromatic end-capping moiety, as described herein.

In some of any of the embodiments described herein, the aromatic end-capping moiety is Fmoc.

In some of any of the embodiments described herein, at least one, or at least a portion, or each structure in the composition is formed, and is made, of a plurality of end-capping modified aromatic amino acids as described herein in any of the respective embodiments.

In some of any of the embodiments described herein, at least one, or at least a portion, or each structure in the composition is formed, and is made, of a plurality of end-capping modified aromatic dipeptides (in which at least one, preferably both, of the amino acid residues is an aromatic amino acid residue).

In some of any of the embodiments described herein, at least one, or at least a portion, or each structure in the composition is formed, and is made, of a plurality of aromatic amino acids.

In some of any of the embodiments described herein, the aromatic amino acid is phenylalanine.

In some of any of the embodiments described herein, at least one, or at least a portion, or each structure in the composition is formed, and is made, a plurality of phenylalanine molecules.

The phrase “aromatic dipeptide” describes a peptide composed of two amino acid residues, at least one, and preferably both, being an aromatic amino acid as defined herein.

The phrase “end-capping modified dipeptide”, as used herein, refers to a dipeptide as described herein which has been modified at the N(amine)-terminus and/or at the C(carboxyl)-terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

In a preferred embodiment of the present invention, the end-capping modified dipeptides are modified by an aromatic (e.g. Fmoc) end-capping moiety.

The end-capping moieties described herein for N-terminus modification can also be utilized for providing an amine-modified aromatic amino acid as described herein.

In some of any of the embodiments described herein, a self-assembled structure (e.g., a hydrogel) as described herein in any of the respective embodiments, is formed by contacting the plurality of peptides as described herein, and a concentration of the peptides (according to any of the respective embodiments described herein) in the solution (according to any of the respective embodiments described herein) is no more than 20 mg/ml (e.g., ranges from 1 to 20 mg/ml or from 5 to 20 mg/ml, including any intermediate values and subranges therebetween), or no more than 15 mg/ml (e.g., ranges from 1 to 15 mg/ml or from 5 to 15 mg/ml or from 10 to 15 mg/ml, including any intermediate values and subranges therebetween), or no more than 10 mg/ml (e.g., ranges from 1 to 10 mg/ml or from 2 to 10 mg/ml or from 5 to 10 mg/ml, including any intermediate values and subranges therebetween), or no more than 7.5 mg/ml (e.g., from 1 to 7.5 mg/ml or from 2.5 to 7.5 mg/ml), and is optionally about 5 mg/ml.

Without being bound by any particular theory, it is believed that such concentrations of no more than 7.5 mg/ml are associated with relatively homogeneous hydrogels which do not inhibit diffusion of compounds, e.g., reactants in an oxygen-sensitive reaction such as described herein.

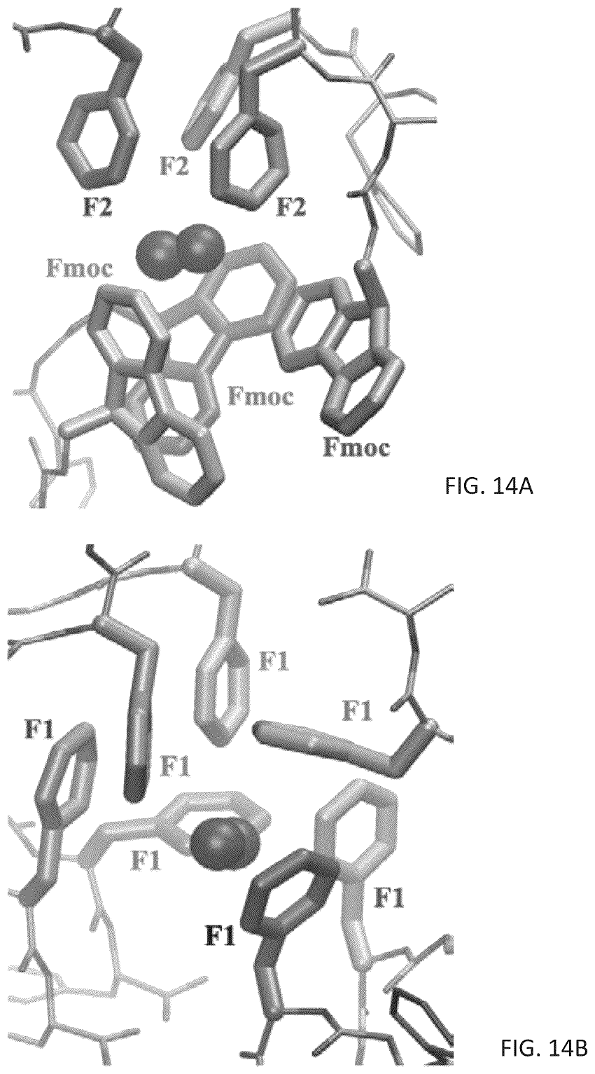

According to some of any of the embodiments described herein, the self-assembled structure is capable of interacting with oxygen such that the oxygen is adjacent to an aromatic ring of each of at least three aromatic amino acid residues, as depicted, for example, in FIGS. 14A-B and is discussed in further detail in the Examples section that follows.

According to some of any of the embodiments described herein, the self-assembled structure is capable of absorbing oxygen in an amount of at least 0.02 mg oxygen per mg of the self-assembled structure, for example, in an amount that ranges from 0.02 to 1, or from 0.02 to 0.1, mg oxygen per mg of the self-assembled structure. In some embodiments, the capability of absorbing oxygen is determined as described herein in the Examples section that follows, or by any other method known in the art.

Controlling Oxygen Concentration:

According to an aspect of some embodiments of the invention, there is provided a method of controlling a concentration of free oxygen in an environment, the method comprising contacting the environment with a self-assembled structure as described herein in any of the respective embodiments and any combination thereof. The self-assembled structure is such that is capable of interacting with oxygen.

Herein throughout, the term “environment” refers to a location or substrate comprising any substance or combination of substances, including solids and fluids (liquids and/or gases).

According to some of any of the respective embodiments described herein, the environment is a fluid environment (e.g., comprises a liquid and/or a gas).

Herein throughout, the phrase “interacting with oxygen” encompasses physical interactions such as absorption, adsorption and/or entrapment of molecular oxygen (O₂), and/or chemical interactions such as, for example, hydrophobic interactions, electrostatic interactions, and/or van der Waals interactions. In some embodiments, the capability of interacting with oxygen is as described herein in any of the respective embodiments, In some embodiments, the capability of interacting with oxygen is determined as described herein in the Examples section that follows, or by any other method known in the art.

By “controlling” oxygen concentration in an environment it is meant effecting a change in oxygen level or concentration in an environment, and encompasses, for example, reducing oxygen concentration in an environment, elevating oxygen level or concentration in an environment, transporting oxygen from one environment to the other, preventing or reducing oxygen contact with an environment, and so forth.

According to some embodiments of the present invention there is provide a method of reducing a concentration of free oxygen in an environment, the method comprising contacting the environment with a self-assembled structure as described herein in any of the respective embodiments and any combination thereof. The self-assembled structure is such that is capable of interacting with oxygen, as described herein.

Reducing a concentration of oxygen according to embodiments of the invention may optionally be effected in anaerobic hoods, gloveboxes and/or clean rooms; in food (and beverage), drug and/or cosmetic product preservation refrigerators, containers and/or packaging; and to preserve devices with oxidizable (e.g., metallic) components, such as electronic devices (e.g., computers), engines (e.g., combustion engines), jewels, and/or cleaning devices (e.g., washing machines, dishwashers). Reducing a concentration of free oxygen can also be effected during manufacturing processes in which anaerobic conditions are desirable, such as rubber materials, or any other process that involves an oxygen-sensitive substance and/or oxygen-sensitive reaction, such as described herein.

In exemplary embodiments, there are provided processes of manufacturing articles, devices or products which include an oxygen-sensitive substance and/or which require anaerobic conditions, which are effected by performing the processes in the presence of a self-assembled structure as described herein in any of the respective embodiments.

In exemplary embodiments, the process is of manufacturing an article, device or product that comprises an oxidizable metallic component. In exemplary embodiments, the process is such that requires anaerobic conditions, for example, a process of manufacturing a rubbery material or of manufacturing an article, device or product that comprises a rubbery material, such as, for example, a process manufacturing tiers. Any other processes that require anaerobic conditions are contemplated.

Such processes can be effected, for example, by incorporating the self-assembled structures as described herein in a reaction medium or reaction container, in which one or more steps of the manufacturing process is/are effected.

In exemplary embodiments, there are provided articles-of-manufacturing comprising an oxygen-sensitive substance and a self-assembled structure as described herein in any of the respective embodiments. Exemplary such articles-of-manufacturing comprise anaerobic hoods, gloveboxes and/or clean rooms; food, beverage, drug, and cosmetic products; food, beverage, drug, and cosmetic preservation refrigerators, containers and/or packaging; and products and devices with oxidizable metallic components, such as electronic devices (e.g., computers), engines (e.g., combustion engines), jewels, cleaning devices (e.g., washing machines, dishwashers); and products and devices with oxidizable rubbery components.

According to some embodiments of the present invention, the environment is or comprises an animate substrate which is susceptible to oxygen damage, and the method is for reducing oxygen concentration or level in or on such a substrate. According to some of these embodiments, the method is effected by contacting such a substrate with a product or article-of-manufacturing that comprises the self-assembled structure as described herein in any of the respective embodiments. An exemplary environment according to these embodiments comprises skin and/or mucosal cells or tissue, and the method is effected by contacting the skin tissue or cell with the self-assembled structure as described herein in any of the respective embodiments or with a product (e.g., cosmetic or cosmeceutical formulation or product) containing same, to thereby reduce oxygen concentration in or on the skin or mucosal cells or tissue.

According to some embodiments of the present invention there is provided a cosmetic product or formulation that comprises a self-assembled structure as described herein in any of the respective embodiments. In some of these embodiments, the cosmetic or cosmeceutical formulation or product is for use in controlling, preferably reducing, oxygen concentration in or on a skin or mucosal tissue of a subject in need thereof. In some of these embodiments, the cosmetic or cosmeceutical formulation or product is for use in reducing or preventing oxygen damage for skin and/or mucosal cells or tissue in a subject.

According to some embodiments of the present invention there is provided a method of controlling (e.g., reducing) oxygen concentration in or on a skin and/or mucosal cell or tissue of a subject, the method comprising contacting the cell or tissue with a self-assembled structure as described herein or with a formulation or product containing same.

According to some embodiments of the present invention there is provided a method of reducing or preventing oxygen damage in or on a skin and/or mucosal cell or tissue of a subject, the method comprising contacting the cell or tissue with a self-assembled structure as described herein or with a formulation or product containing same.

According to an aspect of some embodiments of the invention, there is provided a method of transporting oxygen from a first environment to a second environment (e.g., an oxygen-deficient environment). The first environment optionally has a higher oxygen concentration than the second environment (e.g., such that the oxygen gradient between the first and second environment provides free energy to support the oxygen transport).

The method according to this aspect of the invention comprises:
contacting the first environment with a self-assembled structure (according to any of the embodiments described herein relating to a self-assembled structure) to obtain the self-assembled structure having oxygen interacted therewith; and
contacting the second environment with the self-assembled structure having oxygen interacted therewith, thereby releasing oxygen from the self-assembled structure into the second environment.

In some of any of the embodiments relating to a method of transporting oxygen, the method further comprises contacting the first environment with a self-assembled structure obtained by releasing oxygen from the self-assembled structure. Thus, a given self-assembled structure may optionally be used to transport oxygen at least twice, or at least 10 times, or at least 100 times, or at least 1000 times, or more. Such reuse may optionally be used to reduce a concentration of oxygen in the first environment (according to any of the embodiments described herein relating to a method of reducing a concentration of oxygen), the method being facilitated by release of oxygen in the second environment (optionally a low pressure environment generated by pumping gas away) and reuse of the self-assembled structure.

In some of any of the embodiments relating to a method of transporting oxygen, the oxygen is transported to a second environment comprising an aqueous environment, for example, to prevent the aqueous environment from becoming anoxic.

In some of any of the embodiments relating to a method of transporting oxygen, the second environment is characterized by a continuous depletion of oxygen, for example, continuous depletion of oxygen associated with metabolism of a living organism.

The second environment may optionally comprise a tissue, for example, a living tissue in which oxygen is continuously depleted (at least in part) by cell metabolism in the tissue. In some such embodiments, transport of oxygen to the tissue may be, for example, for enhancing healing of tissue damage (e.g., by providing oxygen to cells associated with tissue regeneration and/or to immune cells inhibiting infection by a pathogenic organism).

In some of any of the embodiments relating to a method of transporting oxygen, the method is for enhancing aerobic metabolism in the second environment, for example, aerobic metabolism associated with degradation (e.g., via oxidation) of a waste product (e.g., oxidation of a waste product in contaminated water, such as sewage). Alternatively or additionally, enhancing aerobic metabolism may be to enhance vitality of aerobic organisms in the second environment (e.g., fish in a body of water or aquarium).

In some of any of the embodiments relating to a method of transporting oxygen, the method is for inhibiting growth of an anaerobic organism in the second environment. The anaerobic organism may be, for example, an obligate anaerobe (i.e., an organism harmed by oxygen per se) and/or an organism which is indirectly inhibited by oxygen, for example, due to competition with or predation by an aerobic organism, which is facilitated by the presence of oxygen. Examples of such organisms include, without limitation, pathogenic organisms such as anaerobic bacteria (e.g., in a tissue, according to any of the respective embodiments described herein) and/or organisms (e.g., bacteria and/or algae) which negatively affect water quality (e.g., wherein the method is used to enhance water quality).

In some of any of the embodiments relating to a method of transporting oxygen, the first environment (e.g., air) is characterized by an oxygen partial pressure of at least 0.15 atmospheres, optionally at least 0.2 atmospheres, optionally at least 0.3 atmospheres, optionally at least 0.5 atmospheres, and optionally at least about 1 atmosphere (e.g., substantially pure oxygen).

According to some of any of the respective embodiments described herein, the self-assembled structure is dispersed in a liquid, e.g., an aqueous liquid. Such a liquid may comprise, for example, fibers and/or gel particles comprising the self-assembled structure dispersed in the liquid.

According to some of any of the embodiments described herein relating to a method of transporting oxygen, the self-assembled structure is dispersed in a liquid (according to any of the respective embodiments described herein), and the method comprises effecting flow of the liquid from the first environment to the second environment (and optionally back to the first environment). Such a liquid may optionally have an additional function, such as cooling (e.g., in a cooling system of an electronic device or in an engine radiator) or cleaning (e.g., in a washing machine or dishwasher).

According to an aspect of some embodiments of the invention, there is provided a self-assembled structure according to any of the respective embodiments described herein, having oxygen interacted therewith. In some such embodiments, an amount of oxygen interacted therewith is at least 0.02 mg oxygen per mg of the structure, according to any of the respective embodiments described herein.

While the above-described embodiments relate to controlling oxygen concentration in an environment which is external to the self-assembled structure, a method of controlling free oxygen concentration can also be utilized for controlling, preferably reducing, oxygen concentration in an internal environment relative to the self-assembled structure, that is, an environment contained in the self-assembled structure. In some embodiments, such a method can be utilized for protecting an oxygen-sensitive substance such as described herein from oxygen damage. In some embodiments, such a method can be utilized for performing a reaction which is oxygen-sensitive and/or which utilizes an oxygen-sensitive substance and can be effected by incorporating the oxygen-sensitive substance in the self-assembled structure.

Compositions and Systems:

According to an aspect of some embodiments of the invention, there is provided a composition comprising a substance which is oxygen-sensitive and/or which participates in an oxygen-sensitive reaction, the substance being incorporated in a self-assembled structure as described herein in any of the respective embodiments (e.g., a hydrogel comprising the self-assembled structure). The self-assembled structure is formed of a self-assembled plurality of aromatic peptides (peptides of 2 to 6 amino acid residues in which one or more of the amino acid residues is aromatic, according to any of the respective embodiments described herein).

Herein, the phrases “substance which is oxygen-sensitive” and “oxygen-sensitive substance”, which are used interchangeably, describe a substance which interacts with oxygen (e.g., binds to oxygen in a manner which alters the properties of the substance (e.g., alters an activity and/or functionality of the substance), and/or decomposes in the presence of oxygen) at room temperature (e.g., 25° C.) or at a temperature at which a reaction or process utilizing same is performed.

An oxygen-sensitive substance can be a substance which, when contacted with or exposed to oxygen as described herein (e.g., an oxygen-containing environment such as air), its activity or functionality are reduced by at least 10%, or at least 20%, or at least 30%, or at least 40%, or at least 50%, or more, relative to its activity or functionality when contacted under the same conditions with an anaerobic, or oxygen-depleted, environment.

Reduction in the activity or functionality of the substance can be readily measured by methods known in the art for a respective substance.

An oxygen-sensitive substance can be a substance which, when contacted with or exposed to oxygen as described herein (e.g., an oxygen-containing environment such as air), at least 10%, or at least 20%, or at least 30%, or at least 40%, or at least 50%, or more, of the substance decompose.

Exemplary oxygen-sensitive substances include, but are not limited to, enzymes, as described herein, metal-containing catalysts, catalysts and/or reagents used in processes of manufacturing rubbery materials, and many other substances.

Herein, an “oxygen-sensitive reaction” refers to a chemical reaction which is substantially inhibited by the presence of oxygen (e.g., an aerobic environment) relative to conditions which are identical (e.g., in pressure, temperature, compounds and concentrations thereof) except for the absence of oxygen (e.g., an anaerobic environment). The effect of oxygen may be due, for example, to an interaction with one or more reactant and/or product of the reaction and/or with a substance that promotes or catalyzes of the reaction (e.g., a catalyst or an activator).

Herein, a “substance which participates in an oxygen-sensitive reaction” may optionally be a reactant (e.g., a substance which is chemically altered by the reaction) and/or a catalyst or activator (e.g., a substance which is not substantially altered upon completion of the reaction) of the reaction.

In some embodiments, the “substance which participates in an oxygen-sensitive reaction” is an oxygen-sensitive substance (as defined herein), e.g., a substance that exhibits reduced reactivity in the reaction in the presence of oxygen, or a substance which decomposes in the presence of oxygen, as described herein.

In some of any of the embodiments described herein, the substance which participates in an oxygen-sensitive reaction is a live cell, such as an anaerobic (e.g., obligatory and/or facultative anaerobic) organism (e.g., bacterium). Thus, for example, a self-assembled structure (e.g., a hydrogel) as described herein may serve as a protective matrix for growth of such cells.

In some of any of the embodiments described herein, the substance which participates in an oxygen-sensitive reaction is a reagent or catalyst used in the manufacturing of a rubbery material (e.g., in a vulcanization process). Thus, for example, a self-assembled structure (e.g., a hydrogel) as described herein may serve as a protective matrix for such a reagent or catalyst and the manufacturing can be effected under aerobic conditions.

In some of any of the embodiments described herein, the oxygen-sensitive substance is a reagent or catalyst used in the manufacturing of a rubbery material (e.g., in a vulcanization process). Thus, for example, a self-assembled structure (e.g., a hydrogel) as described herein may serve as a protective matrix for such a reagent or catalyst and the manufacturing can be effected under aerobic conditions.

In some of any of the embodiments described herein, the oxygen-sensitive substance is an oxygen-sensitive organometallic catalyst. Thus, for example, a self-assembled structure (e.g., a hydrogel) as described herein may serve as a protective matrix for such a catalyst and a reaction catalyzed by the catalyst can be effected under aerobic conditions.

In some of any of the embodiments described herein, the substance which participates in an oxygen-sensitive reaction is an enzyme.

In some such embodiments, the enzyme interacts with oxygen (e.g., at room temperature and/or atmospheric pressure) in a manner which reduces the catalytic ability of a reaction catalyzed by the enzyme, thereby rendering the reaction oxygen-sensitive. Such an enzyme is also referred to herein interchangeably as an “oxygen-sensitive enzyme”.

Oxygen-sensitive enzymes may comprise a transition metal such as iron, for example, in the form of one or more iron-sulfur cluster and/or di-iron (e.g., di-iron azadithiolate) center. In some embodiments, oxygen sensitivity is associated with reaction of oxygen with such a metal, e.g., interfering with reduction and/or oxidation of the metal.

Examples of oxygen-sensitive enzymes include, without limitation, hydrogenases, nitroreductases, and nitrogenases and other enzymes that participate in redox reactions. In some of any of the embodiments described herein, the enzyme comprises an [FeFe]-hydrogenase, for example, a Chlamydomonas reinhardtii [FeFe]-hydrogenase.

In some of any of the embodiments described herein, the composition further comprises an electron transfer mediator, which is optionally conjugated to at least a portion of the aromatic molecules (according to any of the respective embodiments described herein).

Herein, the phrase “electron transfer mediator” refers to a molecule (or moiety of a molecule, or ion of a salt) which facilitates electron transfer by reversibly accepting and donating electrons. The electron transfer mediator has both a relatively stable reduced form and a relatively stable oxidized form in order to be capable of cycling between both such states.

Preferably, at least one relatively stable reduced form and at least one relatively stable oxidized form differ by a single electron, such that the mediator can facilitate transfer of single electrons. Examples of structures suitable for mediating transfer of single electrons (e.g., having a stabilized form generated by acceptance or donation of a single electron, such as a stable radical anion or radical cation) include, without limitation, transition metals and conjugated pi-electron systems (e.g., such as polycyclic aromatic systems and conjugated aromatic rings), especially pi-electron systems comprising heteroatoms (e.g., heteroaryl).

The electron transfer mediator is optionally soluble in a hydrogel, e.g., in an aqueous medium forming the hydrogel. Solubility of the mediator may enhance electron transfer, for example, by enhancing diffusion of the mediator between an electron donor and electron acceptor.

In some of any of the embodiments described herein, the electron transfer mediator comprises a viologens. Methyl viologen (also known in the art as “paraquat”) is an exemplary viologen, e.g., in a form of a chloride salt.

Herein, the term “viologen” refers to any compound having a substituted or unsubstituted bipyridine (preferable 4,4′-bipyridine) structure (including salts thereof), for example, wherein one or both nitrogen atoms are substituted by alkyl (e.g., methyl), alkenyl, alkynyl, cycloalkyl, aryl, heteroaryl and/or heteroalicyclic so as to form a positively charged tetravalent nitrogen atom.

In some of any of the embodiments described herein, a concentration of electron transfer mediator (e.g., viologen) according to any of the respective embodiments is at least 0.1 mM, optionally in a range of from 0.1 to 20 mM, optionally in a range of from 0.1 to 5 mM, and optionally in a range of from 0.1 to 2 mM.

In some of any of the embodiments described herein, a concentration of electron transfer mediator (e.g., viologen) according to any of the respective embodiments is at least 0.3 mM, optionally in a range of from 0.3 to 20 mM, optionally in a range of from 0.3 to 5 mM, and optionally in a range of from 0.3 to 2 mM. In some embodiments, the concentration of electron transfer mediator (e.g., viologen) is about 1 mM.

According to an aspect of some embodiments of the invention, there is provided a compound comprising a peptide, preferably a peptide of from 2 to 6 amino acid residues, and an electron transfer mediator moiety attached (e.g., covalently) to a chemically compatible moiety of the peptide, optionally a C-terminus of the peptide, such that the covalent attachment does not interfere with the self-assembly. The electron mediator moiety may optionally be a residue of an electron transfer mediator according to any of the respective embodiments described herein.

In some of any of the embodiments described herein, the peptide comprises at least one aromatic amino acid, for example, a peptide according to any of the respective embodiments described herein (e.g., in the section pertaining to self-assembling structures).

Attachment of the peptide to the electron transfer mediator moiety is optionally via condensation (e.g., of an electron transfer mediator), such that a C-terminus of the peptide of the compound has the formula —C(═O)-A, wherein A is the electron transfer mediator moiety, which may optionally comprise or consist of a residue of an electron transfer mediator according to any of the respective embodiments described herein.

Exemplary Applications:

A composition of any of the respective embodiments described herein may optionally be in electrical contact with an electrical power source.

According to an aspect of some embodiments of the invention, there is provided a system comprising a composition comprising a self-assembled structure (e.g., a hydrogel) according to any of the respective embodiments described herein, and a current collector configured for being attached to an electrical power source.

In some of any of the embodiments described herein, the system further comprises a reservoir for collecting a gas and/or liquid (and optionally compression of a gas) formed by the substance in the hydrogel (according to any of the respective embodiments described herein), for example, wherein the reservoir comprises an inlet configured for receiving gas and/or liquid from a region containing the composition, and/or a valve configured for allowing entry of gas and/or liquid upon production, and preventing escape of the gas and/or liquid during storage.

The reservoir is optionally configured for being attachable to a device (e.g., fuel cell) which utilizes the gas and/or liquid (e.g., H₂).

According to an aspect of some embodiments of the invention, there is provided a method of oxidizing and/or reducing a substrate, the method comprising contacting the substrate with a composition according to any of the respective embodiments described herein. In some of any of the aforementioned embodiments, the method further comprises applying electric power to the composition.

Examples of suitable substrates include, without limitation, water and air (or any other gas comprising N₂).

In some of any of the embodiments described herein, the method is effected in a presence of O₂ (outside but adjacent to the composition) at a partial pressure of at least 0.002 atm (e.g., from 0.002 to 0.25) atm, optionally at least 0.02 atm (e.g., from 0.02 to 0.25) atm, and optionally about 0.21 atm (a typical partial pressure in air at atmospheric pressure).

Without being bound by any particular theory, effecting a method in the presence of substantial amount of O₂ advantageously reduces or even eliminates a need to remove O₂ (e.g., by pumping or other means) in order to protect the reaction.

In some of any of the embodiments described herein relating to a system and/or method for producing a gas and/or liquid, the substance comprises a hydrogenase and the gas comprises H₂ (e.g., produced from water). The H₂ is optionally for use as a fuel in a fuel cell, e.g., prepared at a purity and/or pressure suitable for such a use. In some embodiments, the method comprises, in addition to producing H₂, further using the H₂ as a fuel (e.g., oxidizing the H₂), for example, in a fuel cell.

In some of any of the embodiments described herein relating to a system and/or method for producing a gas and/or liquid, the substance comprises a nitrogenase and the gas and/or liquid comprises ammonia (e.g., in a form of a gas and/or liquid) and/or a salt thereof (e.g., produced from N₂).

According to other aspects of some embodiments of the present invention, the composition as described herein is usable in any process that utilize an oxygen-sensitive substance or an oxygen-sensitive reaction, which are effected by incorporating the substance as described herein in a self-assembled structure as described herein and performing the process in an oxygen-containing (aerobic) environment.

According to other aspects of the present invention there are provided articles-of-manufacturing or products that comprise a composition as described herein, in which an oxygen-sensitive substance is incorporated in a self-assembled structure as described herein. Exemplary, non-limiting, examples of such articles or products include food, beverage, pharmaceuticals, cosmetic products and cosmeceutical products, which contain oxygen-sensitive substances, and preservation devices or containers or packaging thereof; rubber-containing articles or products such as tiers, and preservation devices or containers or packaging thereof; metal-containing articles or devices as described herein and preservation devices or containers or packaging thereof, etc.

According to an aspect of some embodiments of the present invention there is provided a cosmetic or cosmeceutical formulation or product comprising a self-assembled structure as described herein. In some embodiments the formulation or product are for preventing or reducing oxygen damage in skin or mucosal tissue of a subject in need thereof. In some embodiments, the self-assembled structure is included in the formulation or product for prolonging the shelf-live of the formulation or product. In some embodiments, one or more of the ingredients in the formulation or product is an oxygen-sensitive substance. In some embodiments, the self-assembled structure has an oxygen-substance incorporated therein as described herein in any of the respective embodiments.

According to an aspect of some embodiments of the present invention there is provided an article-of-manufacturing or product that comprises a rubbery material which further comprises a self-assembled structure as described herein. In some embodiments, the self-assembled structure is included in the article or product for prolonging the shelf-live of the article or product (e.g., by preventing oxidation or oxidative processes of the rubbery material).

It is expected that during the life of a patent maturing from this application many relevant oxygen-sensitive substances and/or reactions and/or environments will be uncovered and/or developed and the scope of the terms “oxygen-sensitive substance”, “oxygen-sensitive reaction” and “environment” is intended to include all such new technologies a priori.

It is expected that during the life of a patent maturing from this application many relevant oxygen-sensitive enzymes and reactions utilizing such enzymes will be uncovered and/or developed and the scope of the term “oxygen-sensitive enzyme” is intended to include all such new technologies a priori.

As used herein the term “about” refers to ±20%. In some of any of the respective embodiments described herein, the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, oxo, cyano, nitro, azo, azide, sulfonamide, carbonyl, thiocarbonyl, carboxy, thiocarbamate, urea, thiourea, carbamate, amide, epoxide and hydrazine. The hydrocarbon can be an end group or a linking group, as these terms are defined herein.

Preferably, the hydrocarbon moiety has 1 to 20 carbon atoms. Whenever a numerical range; e.g., “1 to 20”, is stated herein, it implies that the group, in this case the alkyl group, may contain 1 carbon atom, 2 carbon atoms, 3 carbon atoms, etc., up to and including 20 carbon atoms.

Herein, the term “alkyl” describes a saturated aliphatic hydrocarbon end group, as defined herein, including straight chain and branched chain groups. Preferably, the alkyl group has 1 to 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. Substituted alkyl may have one or more substituents, whereby each substituent group can independently be, for example, cycloalkyl, alkenyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

Herein, the term “alkenyl” describes an unsaturated aliphatic hydrocarbon end group which comprises at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or unsubstituted. Substituted alkenyl may have one or more substituents, whereby each substituent group can independently be, for example, cycloalkyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

Herein, the term “alkynyl” describes an unsaturated aliphatic hydrocarbon end group which comprises at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or unsubstituted. Substituted alkynyl may have one or more substituents, whereby each substituent group can independently be, for example, cycloalkyl, alkenyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The cycloalkyl group can be an end group, as this phrase is defined herein, wherein it is attached to a single adjacent atom, or a linking group, as this phrase is defined herein, connecting two or more moieties.

The term “aryl” describes an all-carbon monocyclic or fused-ring polycyclic (i.e., rings which share adjacent pairs of carbon atoms) end group (as this term is defined herein) having a completely conjugated pi-electron system. The aryl group may be substituted or unsubstituted. Substituted aryl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. Phenyl and naphthyl are representative aryl end groups.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The heteroaryl group can be an end group, as this phrase is defined herein, where it is attached to a single adjacent atom, or a linking group, as this phrase is defined herein, connecting two or more moieties. Representative examples are pyridine, pyrrole, oxazole, indole, purine and the like.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic may have one or more substituents, whereby each substituent group can independently be, for example, alkyl, alkenyl, alkynyl, cycloalkyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, cyano, nitro, azo, azide, sulfonamide, carbonyl, thiocarbonyl, carboxy, thiocarbamate, urea, thiourea, carbamate, amide, epoxide and hydrazine. The heteroalicyclic group can be an end group, as this phrase is defined herein, where it is attached to a single adjacent atom, or a linking group, as this phrase is defined herein, connecting two or more moieties. Representative examples are piperidine, piperazine, tetrahydrofuran, tetrahydropyran, morpholine and the like.

As used herein, the terms “amine” and “amino” describe both a —NRxRy end group and a —NRx- linking group, wherein Rx and Ry are each independently hydrogen, alkyl, cycloalkyl, aryl, heteroaryl or heteroalicyclic, as these terms are defined herein. When Rx or Ry is heteroaryl or heteroalicyclic, the amine nitrogen atom is bound to a carbon atom of the heteroaryl or heteroalicyclic ring. A carbon atom attached to the nitrogen atom of an amine is not substituted by ═O or ═S, and in some embodiments, is not substituted by any heteroatom.

The amine group can therefore be a primary amine, where both Rx and Ry are hydrogen, a secondary amine, where Rx is hydrogen and Ry is alkyl, cycloalkyl, aryl, heteroaryl or heteroalicyclic, or a tertiary amine, where each of Rx and Ry is independently alkyl, cycloalkyl, aryl, heteroaryl or heteroalicyclic.

The terms “hydroxy” and “hydroxyl” describe a —OH group.

The term “alkoxy” describes both an —O-alkyl and an —O-cycloalkyl end group, or —O— alkylene or —O-cycloalkyl linking group, as defined herein.

The term “aryloxy” describes both an —O-aryl and an —O-heteroaryl end group, or an —O-arylene- linking group, as defined herein.

The term “thiohydroxy” describes a —SH group.

The term “thioalkoxy” describes both an —S-alkyl and an —S-cycloalkyl end group, or —S— alkylene or —S-cycloalkyl linking group, as defined herein.

The term “thioaryloxy” describes both an —S-aryl and an —S-heteroaryl end group, or an —S-arylene- linking group, as defined herein.

The terms “cyano” and “nitrile” describe a —C≡N group.

The term “nitro” describes an —NO₂ group.

The term “oxo” describes a ═O group.

The term “azide” describes an —N═N⁺═N⁻ group.

The term “azo” describes an —N═N-Rx end group or —N═N— linking group, with Rx as defined herein.

The terms “halide” and “halo” refer to fluorine, chlorine, bromine or iodine. In some of any of the respective embodiments, halo is fluoro (e.g., pentahalo is pentafluoro).

The term “phosphate” refers to a —O—P(═O)(ORx)-ORy end group, or to a —O—P(═O)(ORx)-O— linking group, where Rx and Ry are as defined herein, except when referring to a phosphate ion salt such as a calcium phosphate.

The terms “phosphonyl” and “phosphonate” refer to an —P(═O)(ORx)-ORy end group, or to a —P(═O)(ORx)-O— linking group, where Rx and Ry are as defined herein. The term “phosphinyl” refers to a —PRxRy group, where Rx and Ry are as defined hereinabove.

The term “sulfoxide” or “sulfinyl” describes a —S(═O)—Rx end group or —S(═O)— linking group, where Rx is as defined herein.

The term “sulfonyl” describe a —S(═O)₂—Rx end group or —S(═O)₂— linking group, where Rx is as defined herein.

The term “sulfonate” describes a —S(═O)₂—O-Rx or —O—S(═O)₂—Rx end group or —S(═O)₂—O— linking group, where Rx is as defined herein.

The term “sulfate” describes a —O—S(═O)₂—O-Rx end group or —O—S(═O)₂—O— linking group, where Rx is as defined herein.

The terms “sulfonamide” and “sulfonamido”, as used herein, encompass both S-sulfonamide and N-sulfonamide end groups, and a —S(═O)₂—NRx- linking group.

The term “S-sulfonamide” describes a —S(═O)₂—NRxRy end group, with Rx and Ry as defined herein.

The term “N-sulfonamide” describes an RxS(═O)₂—NRy- end group, where Rx and Ry are as defined herein.

The term “carbonyl” as used herein, describes a —C(═O)—Rx end group or —C(═O)— linking group, with Rx as defined herein. The term “aldehyde” herein describes a —C(═O)H end group.

The term “thiocarbonyl” as used herein, describes a —C(═S)—Rx end group or —C(═S)— linking group, with Rx as defined herein.

The terms “carboxy” and “carboxyl”, as used herein, encompasses both C-carboxy and O-carboxy end groups, and a —C(═O)—O— linking group.

The term “C-carboxy” describes a —C(═O)—ORx end group, where Rx is as defined herein. The term “carboxylic acid” describes a —C(═O)—OH end group, or a deprotonated form (—CO₂⁻) or salt thereof.

The term “O-carboxy” describes a —OC(═O)—Rx end group, where Rx is as defined herein.

The term “urea” describes a —NRxC(═O)—NRyRw end group or —NRxC(═O)—NRy- linking group, where Rx and Ry are as defined herein and Rw is as defined herein for Rx and Ry.

The term “thiourea” describes a —NRx-C(═S)—NRyRw end group or a —NRx-C(═S)—NRy-linking group, with Rx, Ry and Rw as defined herein.

The terms “amide” and “amido”, as used herein, encompasses both C-amide and N-amide end groups, and a —C(═O)—NRx- linking group.

The term “C-amide” describes a —C(═O)—NRxRy end group, where Rx and Ry are as defined herein.

The term “N-amide” describes a RxC(═O)—NRy- end group, where Rx and Ry are as defined herein.

The term “carbamyl” or “carbamate”, as used herein, encompasses N-carbamate and O-carbamate end groups, and a —OC(═O)—NRx- linking group.

The term “N-carbamate” describes a RyOC(═O)—NRx- end group, with Rx and Ry as defined herein.

The term “O-carbamate” describes an —OC(═O)—NRxRy end group, with Rx and Ry as defined herein.

The term “thiocarbamyl” or “thiocarbamate”, as used herein, encompasses O-thiocarbamate, S-thiocarbamate and N-thiocarbamate end groups, and a —OC(═S)—NRx- or —SC(═O)—NRx- linking group.

The terms “O-thiocarbamate” and “O-thiocarbamyl” describe a —OC(═S)—NRxRy end group, with Rx and Ry as defined herein.

The terms “S-thiocarbamate” and “S-thiocarbamyl” describe a —SC(═O)—NRxRy end group, with Rx and Ry as defined herein.

The terms “N-thiocarbamate” and “N-thiocarbamyl” describe a RyOC(═S)NRx- or RySC(═O)NRx- end group, with Rx and Ry as defined herein.

The term “hydrazine”, as used herein, describes a —NRx-NRyRw end group or —NRx-NRy-linking group, with Rx, Ry, and Rw as defined herein.

As used herein, the term “epoxide” describes a

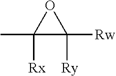

end group or a

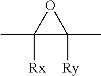

linking group, as these phrases are defined herein, where Rx, Ry and Rw are as defined herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

Agarose (Seakem® LE agarose powder) was obtained from Lonza.

Dimethyl sulfoxide (DMSO) was obtained from Sigma-Aldrich.

Fluorenylmethyloxycarbonyl-diphenylalanine (FmocFF) and tert-butoxycarbonyl-diphenylalanine (BocFF) were obtained from Bachem.

Fluorenylmethyloxycarbonyl-pentafluoro-phenylalanine (Fmoc-F5-Phe) was obtained from Sigma-Aldrich.

Sodium alginate (powder) was obtained from Sigma-Aldrich.

Sample Preparation:

FmocFF stock solution was made by dissolving FmocFF powder in dimethyl sulfoxide (DMSO) at a concentration of 25 mg/ml. Each 1 ml of gel was made by adding 800 μl Tris-HCl buffer (100 mM, pH 7.2) onto 200 μl of FmocFF stock.

BocFF nanotube suspension was prepared by dissolving peptide powder in ethanol at a concentration of 100 mg/ml, diluting this solution in DDW (double-distilled water) to 5 mg/ml, and immediately vortexing for 2-3 seconds.

Fmoc-F5-Phe hydrogel was prepared by dissolving peptide powder in DMSO at a concentration of 100 mg/ml, diluting this solution in DDW to 5 mg/ml, and immediately vortexing for 2-3 seconds.

Solution controls were prepared by adding an equal amount of DMSO to Tris-HCl buffer (100 mM, pH 7.2), in order to maintain similar properties. For agarose samples, Tris-HCl buffer (100 mM, pH 7.2) was heated to 90° C. and Seakem® LE agarose powder was dissolved to 1% (w/v) concentration. The solution was then allowed to cool down to 60° C. with constant stirring, before being transferred to the appropriate vial. Alginate gels were prepared by dissolving 4 mg/ml sodium alginate powder in DDW, and stirring overnight at room temperature. After the sample was transferred to an appropriate vial, a solution of 100 mg/ml CaCl₂ in Tris-HCl buffer (100 mM, pH 7.2) was gently poured over the alginate solution and the whole vial was submerged uncapped in CaCl₂) solution to allow the gel to crosslink overnight.

Methyl Viologen Dye Assay:

Gels or solutions (1 ml) supplemented with 1 mM sodium dithionite and 1 mM methyl viologen (MV) were prepared in 2 ml septum-sealed serum glass vials (Wheaton) in an anaerobic chamber (COY Laboratories). The samples were subsequently sealed and removed from the chamber and exposed to ambient air. The de-colorization of the MV dye was monitored.

O₂ Penetration Assay:

In an anaerobic chamber, anaerobic solutions were used to generate 4 ml gel or solution in Oxvial4™ oxygen sensor vials (PyroScience). The vials were subsequently sealed and removed from the chamber. An optic fiber was connected to the bottom of the vial's sensor and to a FireStingO₂™ oxygen and temperature meter system (PyroScience GmbH). The vials were then exposed to ambient air and O₂ concentration was measured continuously. Measurements were normalized according to ambient temperature.

Mass Spectrometry Analysis:

A QMS (quadrupole mass spectrometer) 200 M1 membrane inlet mass spectrometer (MIMS) (Pfeiffer Vacuum) was used to record headspace gas exchange. Air saturated gels or solutions (2 mL) were prepared in a 5 mL quartz cuvette. The cuvette was fitted into a metabolic chamber (Optical unit ED-101US/MD, Walz) which kept the sample at 25° C. during the experiments. The sample was then sealed and purged with helium for 15 seconds to remove headspace gases, followed by a continuous measurement of N₂, O₂, Ar, and CO₂ with a 3 second interval per mass. All gases were normalized to the H₂ trace to compensate for the continuous removal of the measured gas by the vacuum line.

Enzymatic H₂ Production Assay:

The HydA1 enzyme from Chlamydomonas reinhardtii was expressed and purified from an E. coli Rosetta strain as previously described [Yacoby et al., PLoS One 2012, 7:e35886]. The specific activity of the recombinant protein was 100 μM H₂ per mg HydA1 per minute, which is consistent with reported activity [King et al., J Bacteriol 2006, 188, 2163-2172; Ben Zvi & Yacoby, Int J Hydrogen Energy 2016, 41:17274-17282]. Samples were prepared in 7 ml septum-sealed serum glass vials (Wheaton) under anaerobic atmosphere (97% N₂, 3% H₂). Each 1 ml of FmocFF gel was made by adding 800 μl of Tris-HCl buffer (100 mM, pH 7.2, 1 mM sodium dithionite, 50 nM HydA1) to 200 μl of 25 mg/ml FmocFF stock in DMSO. No further mixing was applied. Since the addition of FmocFF gives a final concentration of 20% DMSO, the solution and agarose samples were supplemented with an equal concentration of DMSO. In the case of agarose, the buffer was heated to 90° C. to dissolve the powder homogenously. To maintain the same conditions, the corresponding samples of solution and FmocFF gel were also heated, and the enzyme was added once all the samples had cooled down to 60° C. FmocFF gelation was carried out after all samples were at room temperature. Following anaerobic preparation, the vials were removed from the anaerobic chamber, and exposed to ambient air for 20 minutes (unless specifically otherwise mentioned). For each set of air-exposed vials, an equal set of anaerobic vials were left sealed. Following air exposure, the vials were resealed, purged with Ar for 1 minute, and supplemented with activity buffer containing 100 mM Tris-HCl, pH 7.2, 1 M NaCl, 20 mM sodium dithionite, and 10 mM methyl viologen. The vials were then incubated at 50° C. in a water bath, while 500 μl of headspace gas samples were drawn at 6-minute intervals. The concentration of H₂ in the vial headspace was measured by a 5890 Series II gas chromatograph (Hewlett-Packard). The residual activity of active HydA1 was determined by comparing the air-exposed samples to the corresponding anaerobic control.

Electron Microscopy:

Scanning electron microscopy (SEM) was performed by Au sputter coating of dried gel samples and imaging in a JSM-IT 100 scanning electron microscope (JEOL) operating at 20 kV. Transmission electron microscopy was done by gently applying 400-mesh copper grids (Electron Microscopy Sciences, Ltd) onto the surface of a gel sample. The grid was subsequently allowed to dry at room temperature overnight. Samples were examined using a 1200EX electron microscope (JEOL), operating at 80 kV.

Rheology Determination:

Rheology was determined using an AR-G2 controlled-stress rheometer (TA Instruments). In order to determine the linear viscoelastic region, oscillatory strain (0.01-100%) and frequency sweep (0.01-100 Hz) tests were performed in parallel plate geometry on 250 μl of freshly prepared hydrogels. The gels were prepared by applying a 50 μl drop of FmocFF stock solution (25 mg/ml in DMSO) directly on the plate, and dropping 200 μl of Tris buffer (100 mM, pH 7.3) onto the stock solution. The geometry was immediately set at a gap size of 0.6 mm, and measurement began. Time sweep oscillatory tests were performed for 2 hours at a constant frequency of 5 Hz and strain of 0.5% to determine G′ and G″, the storage and loss moduli, respectively. All measurements were conducted at room temperature.

Molecular Dynamics (MD) Simulations:

Multiple multi-nanosecond MD simulations were performed to investigate potential binding pockets of O₂ in a complex with a preformed and lightly constrained elementary structural FmocFF fibril. Additional simulations were performed to investigate Ar or CO₂, independently, in complex with an FmocFF fibril, for comparison purposes. All simulations were performed in CHARMM using the CHARMM36 force field, and were analyzed using in-house FORTRAN programs [Brooks et al., J Comput Chem 2012, 32:174-182; Huang & Mackerell, J Comput Chem 2013, 34:2135-2145].

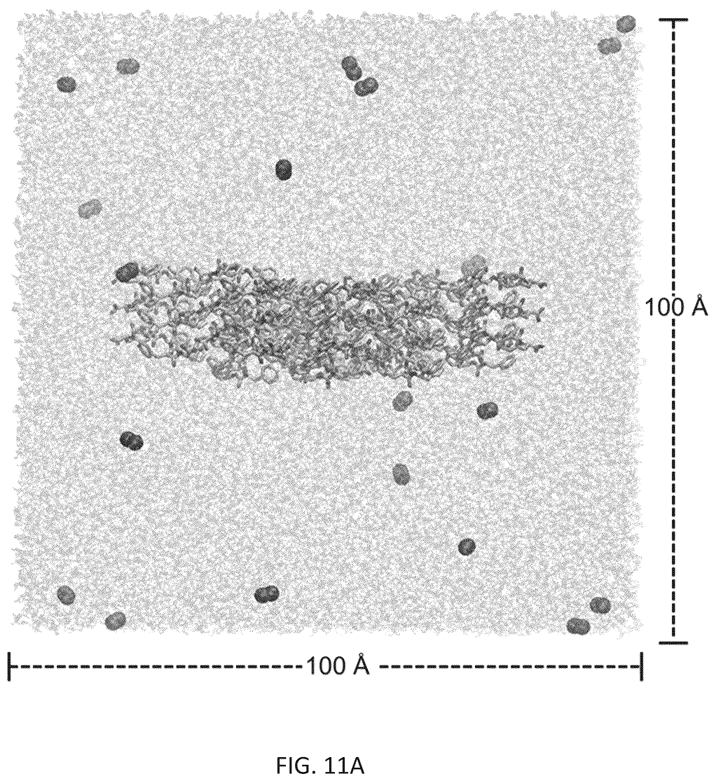

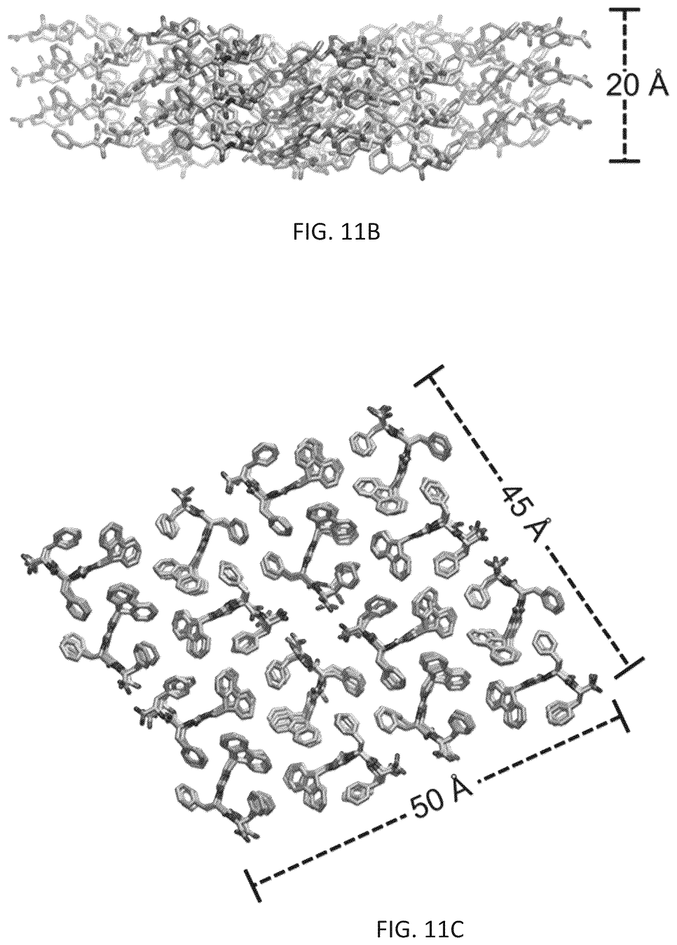

All three systems (Ar, CO₂ and O₂) were simulated in three replicates for a duration of 100 ns per simulation run. The initial structure of the FmocFF fibril corresponded to an experimentally derived crystal structure of the fibril comprising 48 assembled FmocFF peptides [Raeburn et al., Soft Matter 2015, 11:927-935]. The dimensions of the initial structure of the FmocFF fibril were 50×45×20 Å (FIGS. 11A-11C). In their respective simulation systems, 20 O₂, Ar, or CO₂ molecules were initially positioned in a cubic 100×100×100 Å water box surrounding the FmocFF fibril and processed through 2000 steps of Monte Carlo simulations, modeling a O₂, Ar, or CO₂ molecule concentration of 0.035 M. Nevertheless, the initial placement of the gas molecules was performed without any intentional interactions between the molecules and the FmocFF peptides, and inspection of the initial coordinates of all gas molecules showed that no molecules were placed within 10 Å of the Energy minimizations and a 1 ns equilibration stage were performed before executing the three replicate 100 ns MD simulations in each system. The FmocFF peptide backbone atoms and gas molecules were constrained with 1.0 kcal/mol per Å² harmonic constraints. The FmocFF peptide sidechain atoms were constrained with 0.1 kcal/mol per Å² harmonic constraints at their initial position for 100 steps of steepest descent, followed by 100 steps of Adopted Basis Newton-Raphson energy minimization. The systems were subsequently equilibrated for 1 ns. Finally, three replicate MD simulation production runs of 100 ns each were performed for each of the three systems. Three replicate MD simulation runs were preferred for increased sampling over a single run, and to ensure reproducibility. During the production runs, no constraints were imposed on the simulated systems, except for light constraints with 0.1 kcal/mol per Å² harmonic on the FmocFF backbone atoms (N, C, Cα, O). As mentioned in the main text, the constraints were introduced to ensure that the integrity of fibril's assembly is maintained, while providing the ability to study in detail how the investigated molecules interact with the fibril. All energy minimizations and MD simulations were performed using the CHARMM36 force field and periodic boundary conditions in CHARMM [Brooks et al., J Comput Chem 2012, 32:174-182; Klauda et al., J Phys Chem B 2010, 114:7830-7843]. Parameters and topologies for the Fmoc moiety were extracted from Sasselli et al. [Phys Chem Phys 2016, 18:4659-4667]. Parameters and topologies for O₂, Ar, and CO₂ were extracted from Holland et al. [PLoS One 2015, 10:e0122468], Schilling et al. [Phys Chem Phys 2006, 8:1086-1095] and Vanommeslaeghe et al. [J Comput Chem 2009, 3:671-690], respectively. The simulations were not performed using a polarizable force field, due to the unavailability of parameters for all molecules used. The temperature of the simulation systems was maintained at 300 K using the dual Nosé-Hoover thermostat, and the pressure was maintained at 1.0 atm using the Andersen-Hoover barostat [Hoover, Phys Rev A 1985, 31:1695-1697; Li et al., Acta Mater 2003, 51:5711-5742]. The bond lengths of covalently bonded hydrogens were constrained using the SHAKE algorithm [Ryckaert et al., J Comput Phys 1977, 23:327-341]. Upon completion of the MD simulations, the three-replicate simulation trajectories were merged, resulting in a total of 15000 simulation snapshots analyzed per system (5000 simulation snapshots extracted every 0.02 ps per simulation trajectory).

In-house FORTRAN programs were used to structurally analyze the binding of O₂, Ar, and CO₂ molecules to the FmocFF fibril within their respective MD simulation snapshots. The percent of molecules interacting with the FmocFF fibril was calculated as the cumulative number of instances in which the molecule interacts with the fibril divided by the product of the total number of snapshots analyzed and the total number of molecules in the simulation system. A given molecule was considered to be interacting with the FmocFF fibril if any of its atoms were within 4.5 Å of any atom of the FmocFF fibril. The percent molecules bound to the interior of the fibril was also calculated in a similar fashion, with a given molecule considered to be bound to the interior of the fibril if it both interacted with the fibril according to the aforementioned distance criterion, and was less than 10% exposed. The exposure of the molecule was calculated as the solvent accessible surface area divided by the total surface area of a molecule in a given simulation snapshot. Both the solvent accessible surface area and the total surface area of a molecule in a given simulation snapshot were calculated using Wordom with the GEPOL algorithm [Seeber et al., J Comput Chem 2010, 32:1183-1194; Seeber et al., Bioinformatics 2007, 23:2625-2627; Pascual-Ahuir et al., J Comput Chem 1987, 8:778-787]. In addition, the residence time, or the duration for which the molecules remain bound to the fibril irrespective of whether or not the molecule was bound to the interior of the FmocFF fibril was recorded with a resolution of 20 ps. While recording a given molecule's residence time, a “grace period” of 1 ns was allowed, accounting for any instances in which the molecule spontaneously loses contact with the interior of the fibril and subsequently regains contact. The number of binding events per residence time bin is calculated as the number of binding events for a specific residence time duration. Binding events with a residence time less than 1.0 ns long were omitted from the distribution plots.

Analysis of Binding Modes in Molecular Dynamics (MD) Simulations:

In order to analyze the most prominent binding modes of the molecules to the FmocFF fibril within the simulations, a statistical analysis was performed on the interactions formed between the gas molecules and the FmocFF peptides. For each instance in which a molecule was bound to the fibril, the coordinates of the molecule and all peptides with any atom within 4.5 Å of the bound molecule were recorded and classified based on the decomposition of FmocFF peptides into eight groups—(1) the aromatic rings of Fmoc, (2) the pentacyclic ring of Fmoc, (3) the ester group of Fmoc, (4) the sidechain of the first phenylalanine, (5) the backbone of the first phenylalanine, (6) the sidechain of the second phenylalanine, (7) the backbone of the second phenylalanine, and (8) the terminal end of the second phenylalanine. Structures with the same interaction-based classification and the same number of interacting peptides were further decomposed into binding modes using root mean squared deviation (RMSD) based clustering. In the RMSD based clustering, the collected structures were first aligned using the heavy atoms of the interacting FmocFF peptides and subsequently clustered based on the heavy atoms of the peptides using the quality clustering method and a 3.0 Å RMSD cutoff in Wordom [Seeber et al., Bioinformatics 2007, 23:2625-2627; Allouche et al., J Comput Chem 2012, 32:174-182]. The structures grouped into the same cluster were considered to belong to the same binding mode. According to this method, structures containing the same interaction-based classification and with similar geometries are grouped into the same binding mode irrespective of which of the 48 FmocFF peptides within the modeled fibril are interacting with the gas molecule. The binding modes were recorded only if the molecule was bound in the interior of the FmocFF fibril with a residence time of at least 1 ns. Only binding modes occurring in 10% or more of all instances of O₂, Ar, and CO₂ binding to the FmocFF fibril within their respective simulations are reported.

Statistics:

Groups are described herein as value±standard error. Shapiro-Wilk test (α=0.05) was employed to test whether the sample distribution was normal. Variance was analyzed by one-way ANOVA followed by Tukey post hoc test for significant variance between the samples.

### Example 1

### FmocFF Hydrogelation in Buffer Solution

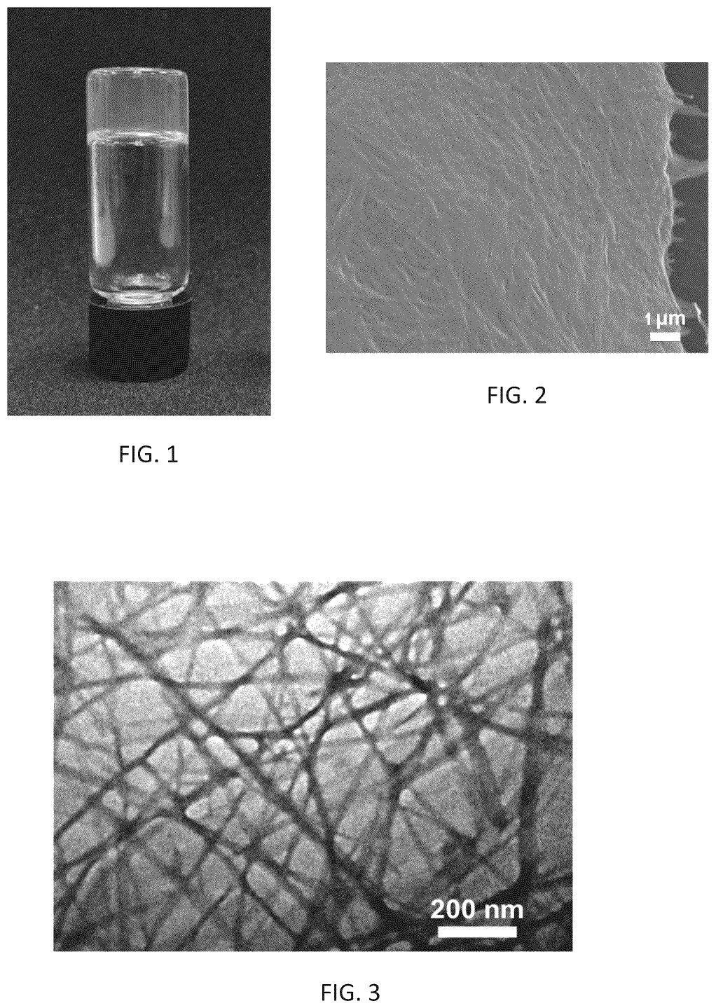

FmocFF hydrogels may be prepared by pH change or solvent switch. In the pH change method, the peptide is dissolved in water supplemented with a base, followed by neutralization using stepwise addition of an acid [Jayawarna et al., Adv Mater 2006, 18:611-614]. This method can expose any dissolved protein to unfavorable conditions. Alternatively, the solvent switch method can be employed, by which the peptide is dissolved in organic solvent, and is then mixed with purified water to trigger self-assembly [Mahler et al., Adv Mater 2006, 18:1365-1370]. While the solvent switch method is suitable for enzyme encapsulation, a modification of the hydrogel from water- to a buffer-based matrix was sought in order to maintain optimal pH and preserve the enzymes' integrity and activity. To this end, FmocFF was dissolved in DMSO (at 25 mg/ml) to produce a solution of monomers, and then diluted with Tris buffer (100 mM, pH 7.3) to a final concentration of 5 mg/ml so as to trigger its self-assembly. This resulted in the formation of a self-supporting, stable 3D hydrogel, shown here in an inverted tube (FIG. 1).

As shown in FIGS. 2 and 3, the hydrogel is composed of nanometric fibrils, as observed by scanning electron microcopy and transmittance electron microscopy, respectively.

This nanofibrillar structure is consistent with previous reports of the FmocFF hydrogel formed in water [Mahler et al., Adv Mater 2006, 18:1365-1370].

The FmocFF hydrogel was further characterized by rheology tests.

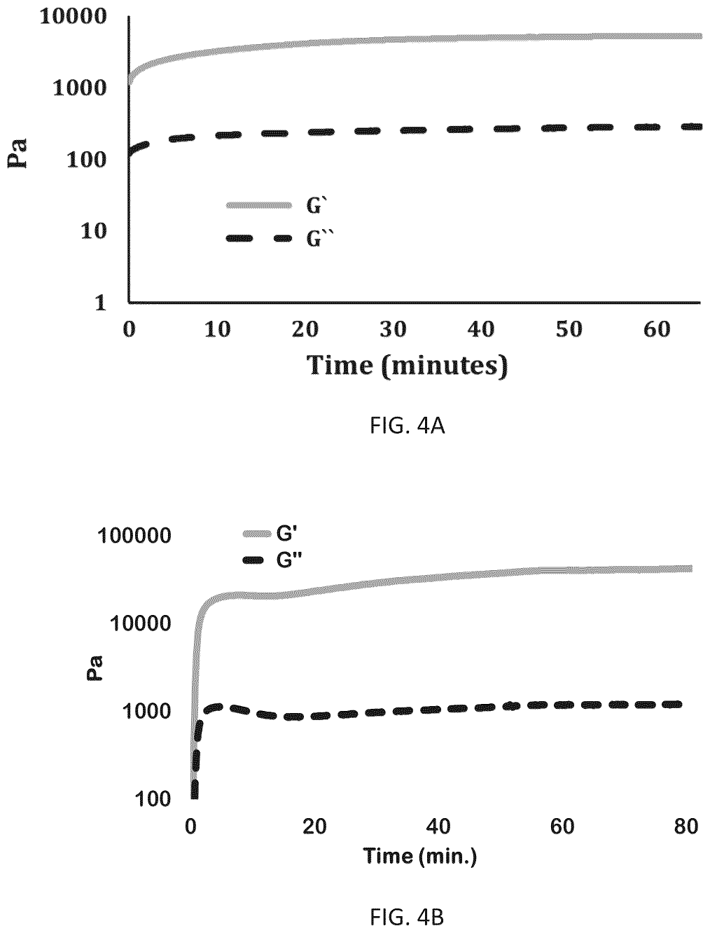

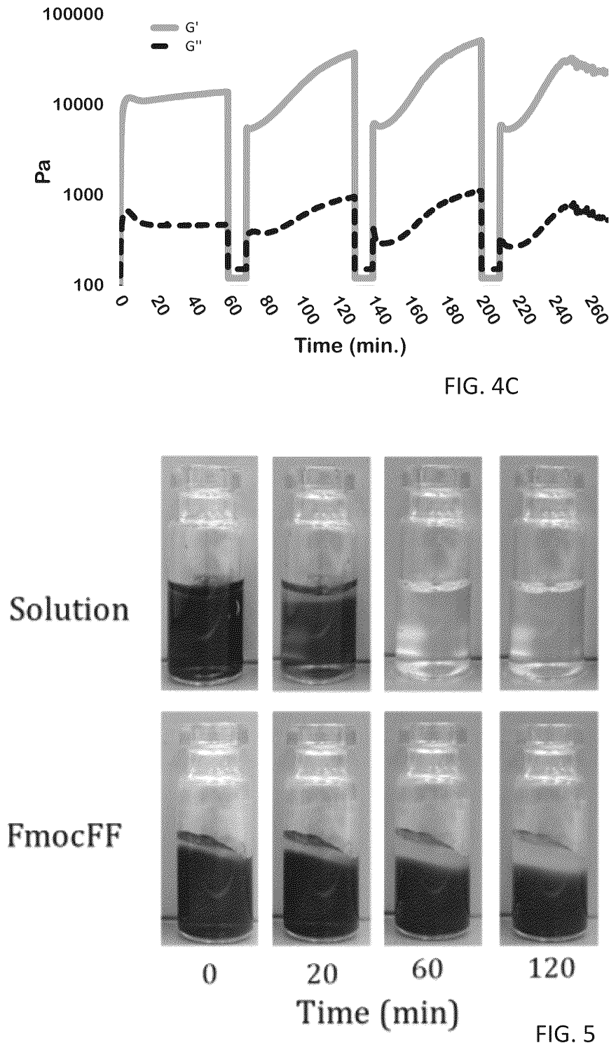

As shown in FIGS. 4A and 4C, the gelation process in Tris buffer occurred rapidly at room temperature, and the resulting hydrogel exhibited remarkable mechanical properties, with a storage modulus (G′) of about 5,000 Pa, and a loss modulus (G″) of about 300 Pa. Gelation occurred within a second, although the maximum storage modulus of about 5,000 Pa, was obtained after 1 hour.

As shown in FIG. 4C, the hydrogel exhibited thixotropic properties.

The gelation process in Tris buffer was notably faster than previously reported gelation in water, and was dependent on Tris concentration (data not shown), suggesting that the increased gelation rate is due to buffer ions acting as nucleation centers for the self-assembly of the FmocFF peptides.

### Example 2

### O₂ Penetration of Exemplary FmocFF Hydrogel

In order to study the kinetics of O₂ penetration into the hydrogel prepared as described in Example 1, samples containing MV (methyl viologen), which reversibly changes color according to oxidation state, were prepared. Under anoxic conditions, MV was reduced by sodium dithionite, to produce a strong violet color. Samples were subjected to gelation by dissolving FmocFF in DMSO, then diluting it into an MV solution, to form an FmocFF 3D-hydrogel with a violet color. The hydrogels were then challenged with ambient air, and the resultant MV bleaching was monitored as MV was oxidized by molecular oxygen.

As shown in FIG. 5, complete bleaching of MV in solution was achieved within less than one hour, indicating thorough penetration of the solution by O₂; whereas bleaching of MV encapsulated in FmocFF hydrogel was observed only at the air-exposed interface.

These results indicate that the FmocFF hydrogel exhibits a remarkable ability to restrict O₂ penetration.

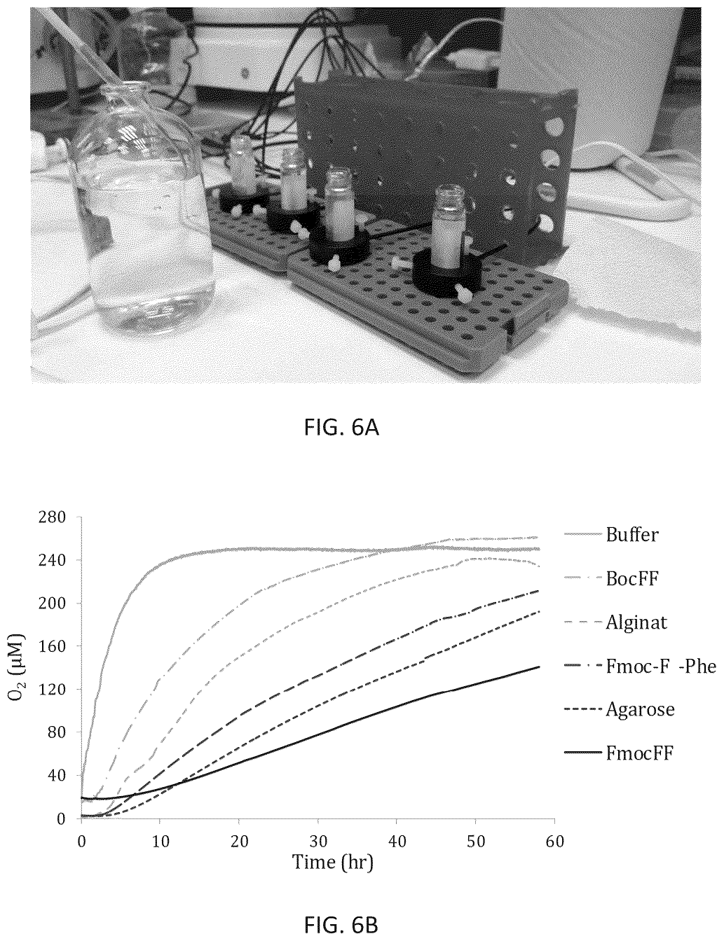

To further investigate this phenomenon, a FireStingO₂™ optical oxygen meter system (PyroScience GmbH), shown in FIG. 6A, was utilized. The probe vials were filled with FmocFF hydrogel under anaerobic conditions. The vials were then exposed to ambient air, and O₂ concentrations near the bottom of the vials were monitored in real time.

As shown in FIG. 6B, the O₂ concentration in a solution increased sharply within minutes of exposure, reaching saturation at 250 μM O₂ after 12 hours; whereas FmocFF hydrogel remained relatively anoxic at 18 μM O₂ for almost 6 hours. The maximal rate of increase in O₂ concentration was calculated from the initial linear phase and was 34 μM/hour in gel-free, buffer solution, as compared to 2.5 μM/hour in FmocFF hydrogel.

In order to assess how much of this effect is unique to FmocFF hydrogels, and how much is a general effect due to the limited diffusion of small molecules in soft solid material, O₂ penetration into other hydrogels was tested in a similar manner. The tested gels included cross-linked alginate, agarose, and Fmoc-pentafluoro-phenylalanine (Fmoc-F5-Phe), another low molecular weight hydrogelator [Ryan et al., Soft Matter 2010, 6:475-479]. In addition, the contribution of the FF (diphenylalanine) moiety to the interactions of FmocFF with O₂ in the gel was tested by comparison with N-(tert-butoxycarbonyl)-diphenylalanine (BocFF), an N-terminus-modified peptide that self-assembles into elongated fibrils, but does not form a gel [Reches & Gazit, Isr J Chem 2005, 45:363-371].

As further shown in FIG. 6B, FmocFF was markedly superior to all the other hydrogels in reducing penetration of O₂. The maximal rates of O₂ penetration into alginate, Fmoc-F5-Phe, and agarose gels were 7.4 μM/hour, 5.7 μM/hour, and 4.3 μM/hour, respectively, as compared to 2.5 μM/hour in FmocFF hydrogel. As further shown therein, the rate of O₂ penetration into a suspension of BocFF nano-tubes was significantly higher than in the tested hydrogels, albeit slightly reduced compared to solution.

These results indicate that the remarkably low rate of O₂ penetration into FmocFF hydrogel is not common to other gels, or even gels formed from other Fmoc-capped amino acids, such as Fmoc-F5-Phe.

In order to investigate whether the restrictive property of the FmocFF hydrogel relates to a general diffusion mechanism that also applies to gases other than O₂, a membrane inlet mass spectrometer (MIMS) was used to monitor the release rates of different gases from gels and solutions. Samples of gels or solutions were prepared in ambient atmosphere and allowed to reach complete saturation. The samples were then introduced into a sealed MIMS device and the headspace was briefly purged with helium gas. Following headspace purging, the dissolved gases in the sample began diffusing back into the cuvette headspace, where the concentrations of N₂, O₂, Ar, and CO₂ were monitored in real-time.

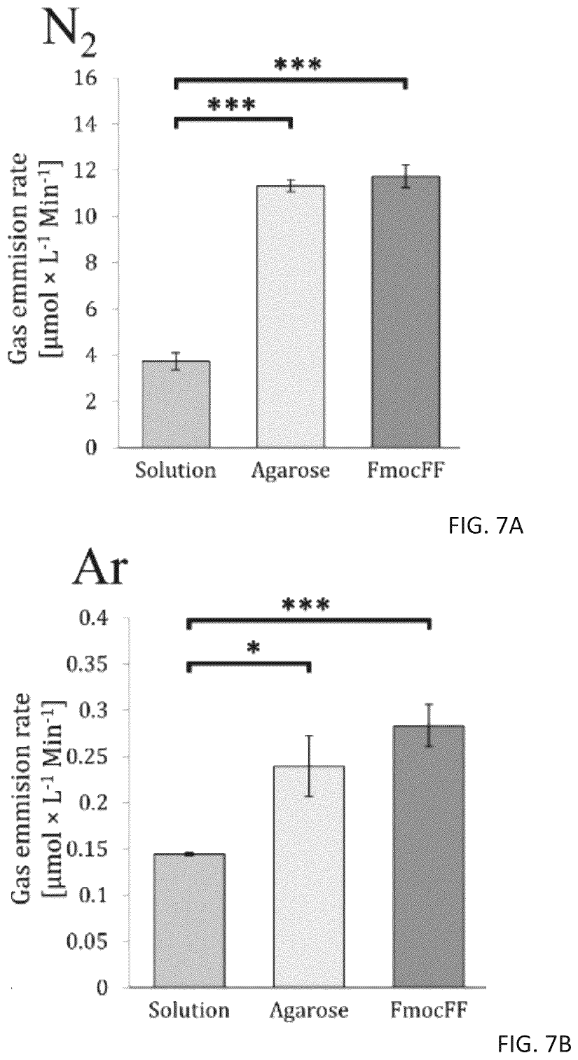

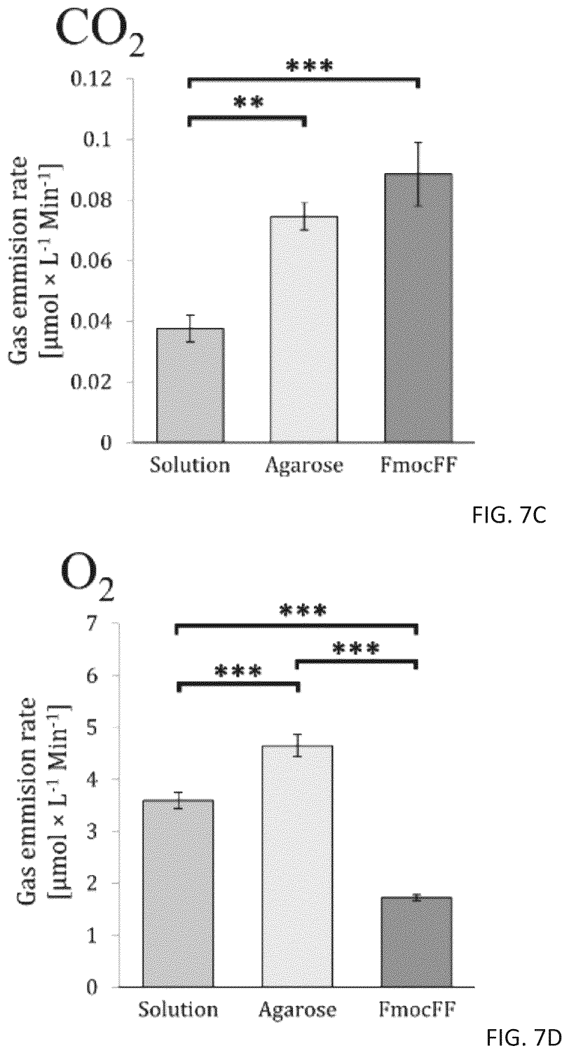

As shown in FIGS. 7A-7D, the rates of gas emission from the gel matrices were significantly higher than those from aqueous solutions, with emission rates for N₂ (FIG. 7A), Ar (FIG. 7B), and CO₂ (FIG. 7C) being about 2-3-fold faster from gels than from solutions, whereas O₂ emission from agarose gel was 30% faster than from the solution (FIG. 7D).

These results may be attributed to a lower solubility of gases in gels, and/or to faster transition to the gas phase associated with heterologous gas nucleation promoted by the high surface area of the nanofibrils [Jones et al., Adv Colloid Interface Sci 1999, 80:27-50].

As further shown in FIG. 7D, 02 emission from FmocFF gels was about 50% slower than O₂ emission from solution, in contrast to the enhanced gas emission observed for O₂ in agarose gels and for N₂, Ar, and CO₂ in both agarose and FmocFF gels.

These results suggest the presence of specific interactions between FmocFF and O₂, and provide further confirmation that O₂ is indeed specifically encaged by the FmocFF fibrils.

Similar results were obtained when agarose and FmocFF gels were prepared with water instead of buffer, and both O₂ penetration and gas emission experiments were repeated.

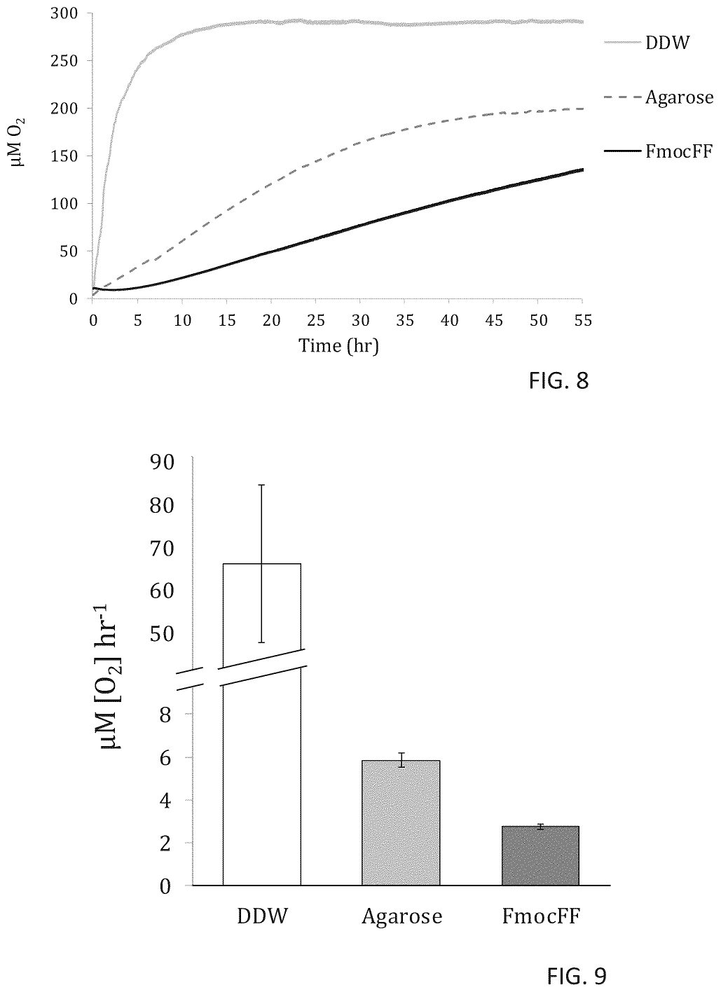

In particular, as shown in FIG. 8, the O₂ concentration in a solution increased sharply within minutes of exposure, reaching saturation at 290 μM O₂ after 12 hours; whereas FmocFF hydrogel remained relatively anoxic at 10 μM O₂ for almost 4 hours, and was markedly superior to agarose hydrogel in reducing penetration of O₂.

As shown in FIG. 9, the maximal rate of increase in O₂ concentration (at the initial linear phase) was 65 μM/hour in gel-free, buffer solution, as compared to 5.8 μM/hour in agarose, and 2.7 μM/hour in FmocFF hydrogel (FIG. 9).

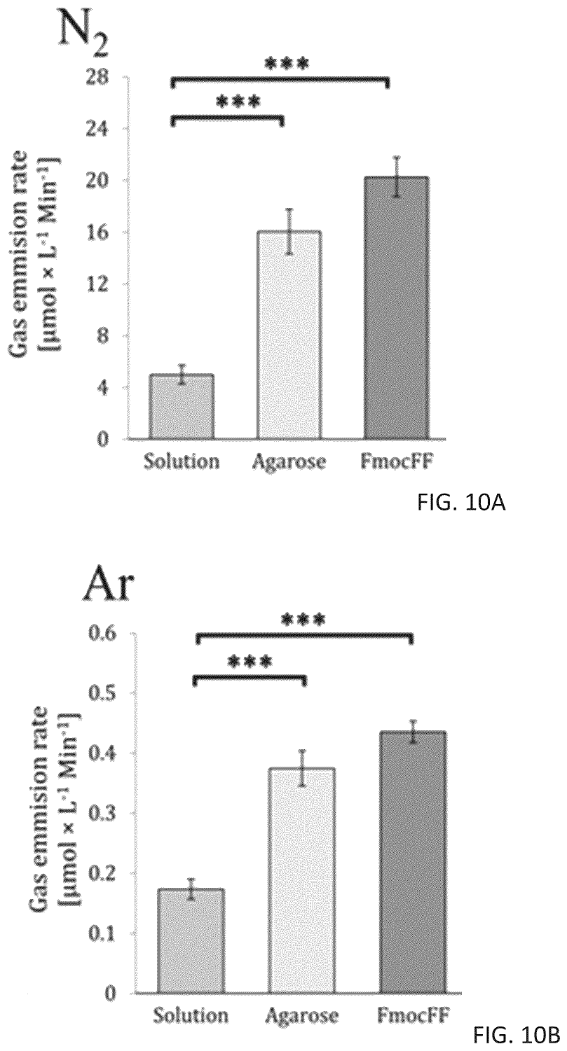

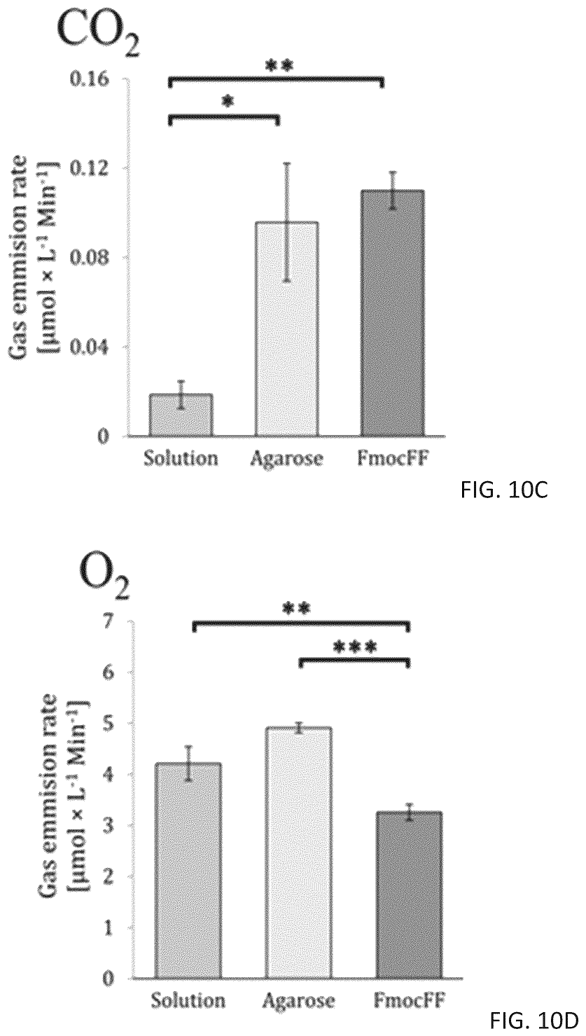

As shown in FIGS. 10A-10D, the rates of gas emission from the gel matrices were generally significantly higher than those from aqueous solutions, with emission rates for N₂ (FIG. 10A), Ar (FIG. 10B), and CO₂ (FIG. 10C) being about 2-5-folds faster from gels than from solutions, and O₂ emission from agarose gel was 20% faster than from the solution (FIG. 10D); whereas O₂ emission from FmocFF gels was about 30% slower than O₂ emission from solution (FIG. 10D).

These results indicate that FmocFF gels prepared with water and FmocFF gels prepared with buffer behave similarly, and confirm that the remarkable behavior of FmocFF gels is obtainable with different hydrogel aqueous phases.

### Example 3

### In Silico Analysis of Interaction Between O₂ and FmocFF Fibrils

In order to shed light onto the mechanism behind the interaction of O₂ and FmocFF hydrogels, multiple in silico multi-nanosecond molecular dynamics (MD) simulations were performed for a lightly constrained FmocFF fibril (as depicted in FIGS. 11A-11C) in the presence of either O₂, Ar, or CO₂ molecules in explicit solvent (water) using CHARMM [Brooks et al., J Comput Chem 2012, 32:174-182] and the CHARMM36 force field [Klauda et al., J Phys Chem B 2010, 114:7830-7843], according to procedures described in the Materials and Methods section hereinabove. Light constraints on the FmocFF backbone heavy atoms of a preformed fibril were introduced to ensure that the integrity of fibril's assembly was maintained. The use of constraints should not be considered to have negatively affected the simulated systems; rather it provided the ability to study in detail how the investigated molecules interact with the fibril. The percentage of gas molecules bound to the fibril were calculated by summing the number of gas molecules within 4.5 Å of any atom of the fibril in each MD snapshot, and dividing by the product of the total number of molecules in the simulation and the total number of simulation snapshots analyzed. The concentration of the molecules within the simulated systems was higher than that used in experiments, which aimed to accelerate the sampling of the molecules around and within the fibril, and is similar to procedures previously described for investigating self-assembly of peptides [Tao et al., Adv Funct Mater 2020, 30:1909614; Chen et al., ACS Nano 2020, 14:2798-2807; Tamamis et al., Biophys J 2009, 96:5020-5029; Wang et al., J Mol Biol 2017, 429:3893-3908].

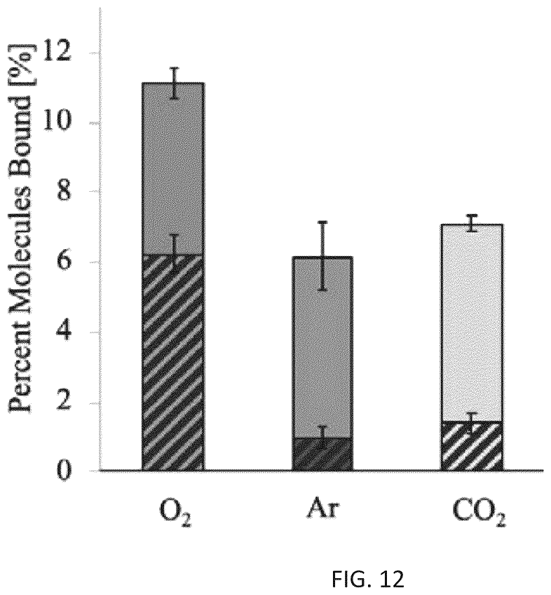

As shown in FIG. 12, the percentage of O₂ molecules bound to the fibril (11.1±0.4%), was considerably higher than the percentage of either Ar atoms (6.2±1.0%) or CO₂ molecules (7.1±0.24%) bound to the fibril, as determined by molecular dynamics simulations. As further shown therein, O₂ molecules bound to the interior of the fibril significantly more frequently (6.2±0.5%) than did either Ar (1.0±0.3%) or CO₂ (2.4±0.3%). In addition, the small values of standard errors are indicative of reproducibility among the three different simulation trajectories per investigated system.

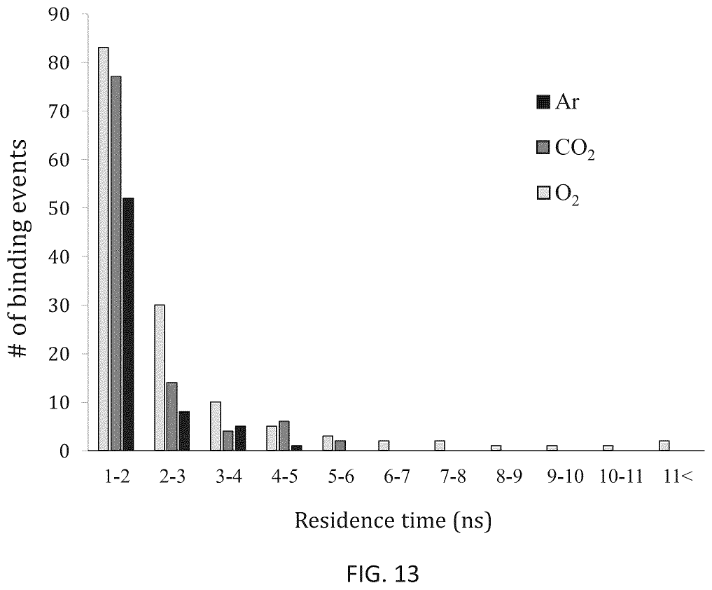

Moreover, as shown in FIG. 13, the binding of O₂ in the interior of the fibril was also associated with longer residence times (i.e., reflecting the duration of binding, as compared to either Ar or CO₂. The higher binding percentage and the longer residence time of O₂ within the interior of the fibril are in line with experimental evidence, and support the hypothesis that O₂ is engaged by the FmocFF fibril.

These results indicate that the inhibition of penetration of FmocFF hydrogels by O₂ is associated with interactions between O₂ and the surface of the hydrogel.

In order to gain additional insight into the mechanism of O₂ binding by FmocFF fibrils, the simulated binding conformations of O₂ within the fibril were analyzed according to procedures described in the Materials and Methods section hereinabove, and two main modes of binding were identified.

As shown in FIG. 14A, the most frequent configuration (referred to as binding mode I), which occurred in 58.9% of cases, involved O₂ being encapsulated in a pocket formed by three Fmoc moieties and three phenylalanine rings, belonging to five individual FmocFF peptides. In this binding mode, two FmocFF peptides interacted with O₂ through the aromatic rings of the individual Fmoc moieties, two through the aromatic rings of phenylalanine-2, and one through both the aromatic rings of the Fmoc moiety and phenylalanine-2.

As shown in FIG. 14B, the second most frequent configuration (referred to as binding mode II), which occurred in 16.2% of all cases, involved O₂ being encapsulated in a hydrophobic pocket formed by seven aromatic rings of phenylalanine-1 from seven individual FmocFF peptides.

Similar analyses performed for Ar and CO₂ observed these molecules in binding mode I for 15.0% and 19.5% of all binding instances, respectively, and in binding mode II for 32.3% and 20.9% of all instances, respectively.

These computational results suggest that O₂ binds preferentially in binding mode I, where it interacts with a binding pocket formed by the three Fmoc moieties and three phenylalanine rings; which is consistent with the experimental results discussed hereinabove showing specificity of the FmocFF interaction with O₂.

### Example 4

### Effect of Hydrogel on Activity of Incorporated Oxygen-Sensitive Enzyme

FmocFF was dissolved in DMSO, and then diluted with Tris buffer at pH 7.2 to trigger its self-assembly, through multiple π-π stacking between aromatic groups together with other weak bonds, such as hydrogen interactions. This resulted in the formation of a self-supporting, stable 3D hydrogel, as shown here in FIG. 1. The final concentration of FmocFF (20 mg/ml) was selected in order to facilitate formation of a dense 3-dimensional nanofiber network, which may enhance the protective effect of gel.

In order to assess the suitability of this hydrogel for encapsulation of an exemplary oxygen-sensitive enzyme, [FeFe]-hydrogenase (HydA) from the green algae Chlamydomonas reinhardtii, 02 penetration through the gel was tested using a FireStingO₂™ optical oxygen meter system. Specialized FireSting™ probe vials were filled under anaerobic conditions with 3 ml of either Tris buffer (pH 7.2) or buffered FmocFF hydrogel. The system's fiber optics were attached near the bottom of the vials. The vials were then exposed to ambient atmosphere and O₂ concentration was measured in real-time for 72 hours, as depicted in FIG. 6A.

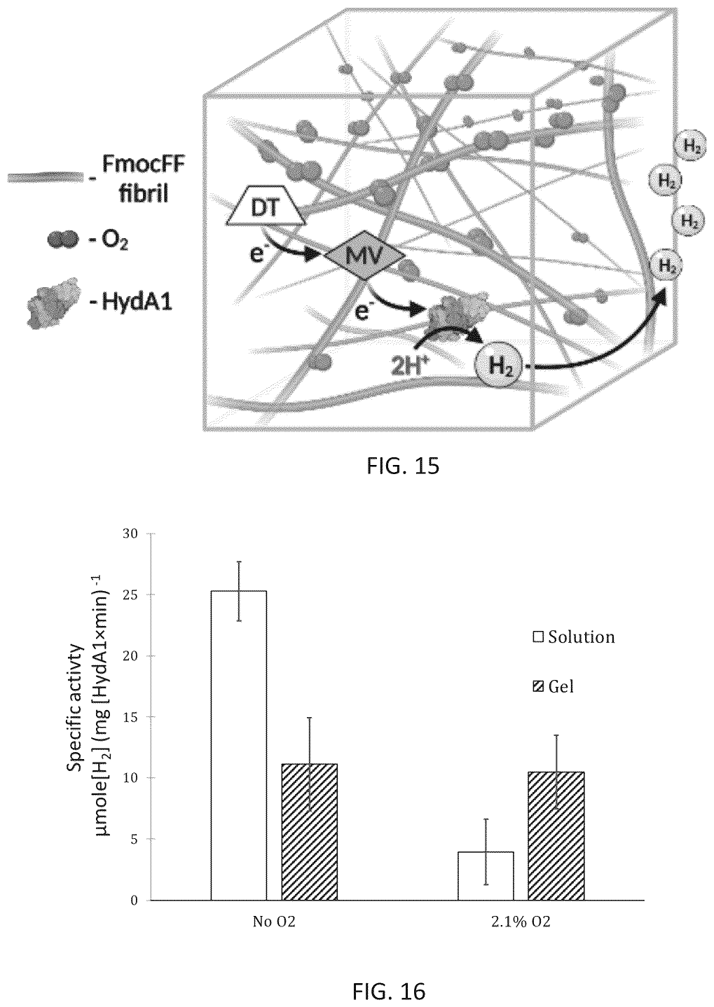

Enzymatic activity in hydrogels was then determined by measuring H₂ gas production under anaerobic and aerobic conditions, as schematically depicted in FIG. 15. The O₂-sensitive enzyme HydA1 is encapsulated in an FmocFF hydrogel, FmocFF fibrils bind O₂ during the exposure period, preventing interaction of O₂ with HydA1 and maintaining its enzymatic activity; the HydA1 is activated by sodium dithionite (DT) and methyl viologen (MV) as electron donor and mediator, respectively, and the produced H₂ diffuses out of the hydrogel.

In an anaerobic glove box, vials of buffer solution with HydA enzyme were prepared with 2.5 mM sodium dithionite (DT) and 1 mM methyl viologen (MV) added as electron donor and electron transfer mediator, respectively. Half of the vials were subjected to gelation with the addition of FmocFF stock solution in DMSO (final concentration 20 mg/ml). Vials were then sealed, 2.1% O₂ was injected into the headspace, and H₂ production was measured by sampling the headspace for gas chromatography (GC) analysis.

As shown in FIG. 16, although H₂ was produced at a lower rate in an exemplary gel than in solution under anaerobic conditions, the rate of H₂ production in the gel was maintained upon exposure to 2.1% O₂, but decreased 6-fold in solution upon exposure to 2.1% O₂. Thus, at 2.1% 02, the rate of H₂ production was considerably higher in the gel than in solution.

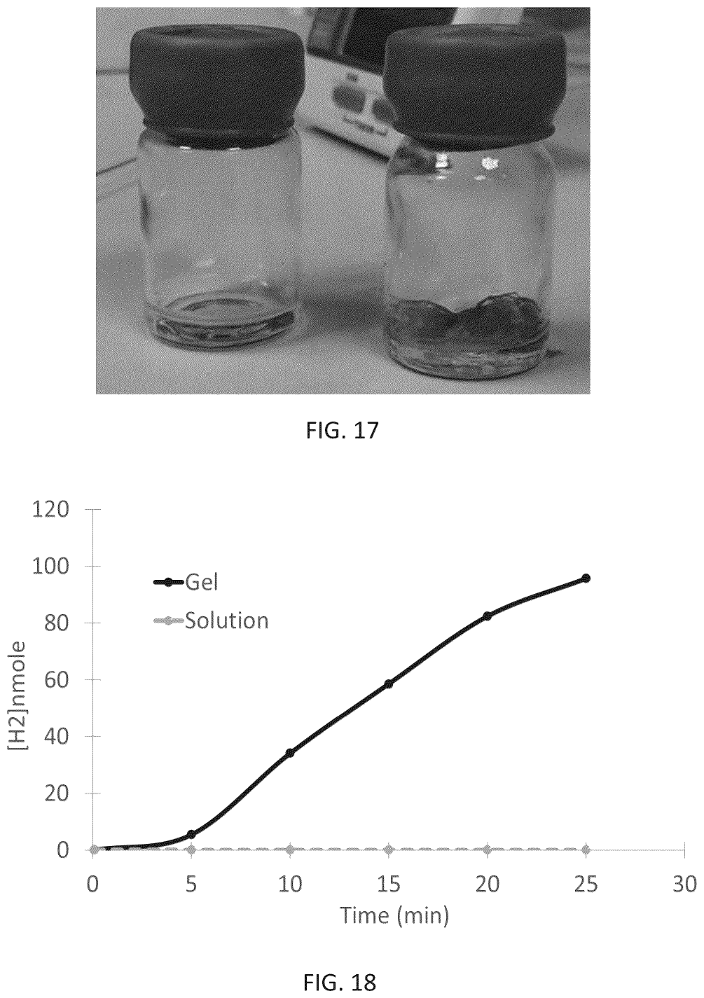

As shown in FIG. 17, the protection by the hydrogel from O₂ was also readily observed using methyl viologen's electrochromism, as the color of methyl viologen was rapidly lost in solution (due to oxidation; see, left vial) but preserved in the hydrogel for the duration of the experiment (over 20 minutes; see, right vial).

The protective effect of FmocFF hydrogels against oxygen was then assessed by exposing HydA-containing hydrogels (or solutions) to a higher concentration of oxygen and for longer durations, i.e. atmospheric air (about 21% oxygen) for 1 hour. Furthermore, in order to ascertain that the protective effect is not simply a result of the reductive capabilities of dithionite and/or methyl viologen, samples were prepared without reducing agents and exposed to ambient air for 1 hour. The vials were then sealed, the headspace purged, and only then was an activation solution of sodium dithionite and methyl viologen injected into the vials, covering the gels or mixed with the HydA solution. The purple color (associated with methyl viologen) was observed to quickly diffuse through the hydrogels.

As shown in FIG. 18, H₂ accumulation in the headspace, in an approximately linear pattern, was detected in the gels but not in solution.

These results indicate that the 1-hour exposure to air eliminated all the HydA in solution, whereas HydA in the exemplary gel was protected from air, while allowing transfer of redox equivalents and H₂ through the gel.

In order to enhance H₂ production and reproducibility of the hydrogel system, hydrogels were formed also with FmocFF concentrations of 10 or 5 mg/ml. It was hypothesized that the opaque and non-uniform appearance of the abovementioned hydrogels (with 20 mg/ml FmocFF) was associated with relatively large standard deviations observed in measurements of enzyme activity.

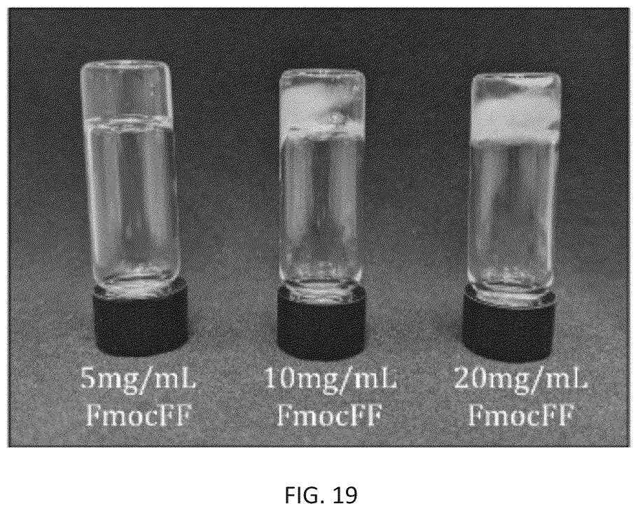

As shown in FIG. 19, reduction of FmocFF concentration to 5 mg/ml resulted in a clear and homogenous gel, associated with slower gel assembly.

As in previous experiments, the samples were made in an anaerobic glove box and exposed to ambient air for 20 minute, after which the vials were sealed, headspace purged, sodium dithionite and methyl viologen added, and H₂ production was measured in regular time intervals. Residual HydA activity was calculated (as a percentage) by comparing enzymatic H₂ production rate in the gels to that of solutions under strict anaerobic conditions.

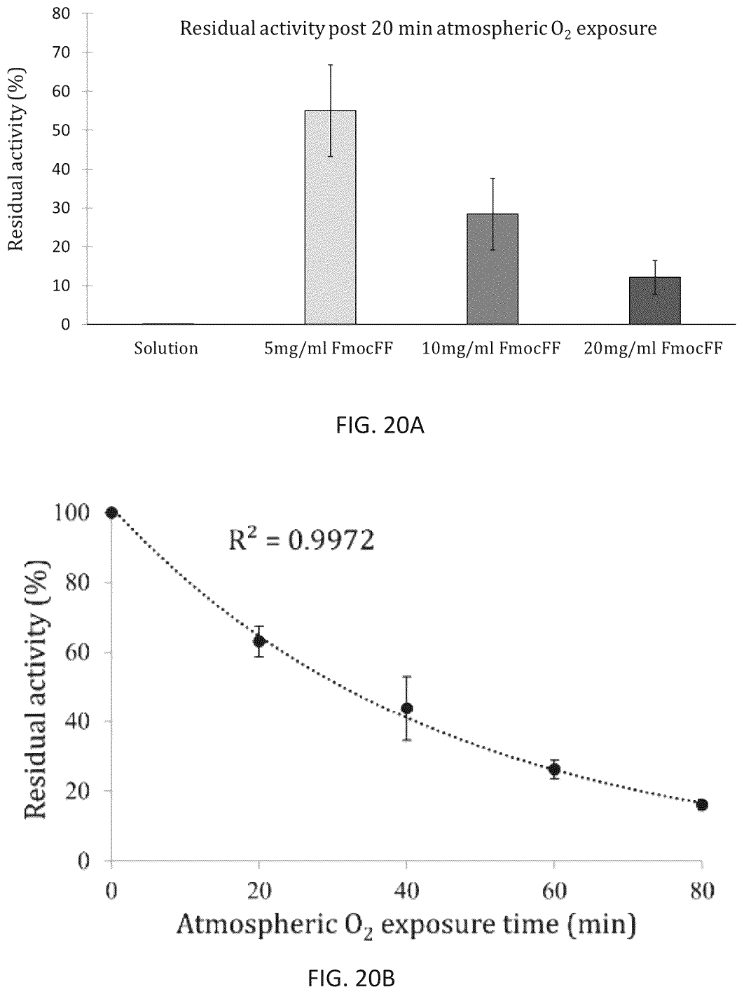

As shown in FIG. 20, gels prepared with 5 mg/ml FmocFF produced more uniform results, with standard deviation reduced to 20% of the average, as compared with gels prepared with 10 or 20 mg/ml FmocFF (e.g., standard deviation for 20 mg/ml was about 50% of the average). As further shown therein, the 5 mg/ml gel also offered significantly better protection of the enzyme from O₂, as compared to 10 and 20 mg/ml gels, while still allowing diffusion of redox equivalents and H₂ gas across the gel.

The protective effect of FmocFF hydrogel was further assessed by comparison with other known hydrogels. For this purpose, agarose gel was selected. Tris buffer was heated to 90° C. for all samples, and agarose powder was dissolved in the hot buffer and allowed to cool to 60° C. before adding the enzyme. Solution and FmocFF samples were prepared in the same manner, but FmocFF peptides were added once the samples reached room temperature. Solution, agarose, and FmocFF gels were then exposed to ambient air for 20 minutes, sealed and the headspace was purged. Activity solution of sodium dithionite and methyl viologen was added, and hydrogen production was determined at various time points by GC analysis of the vial headspace.

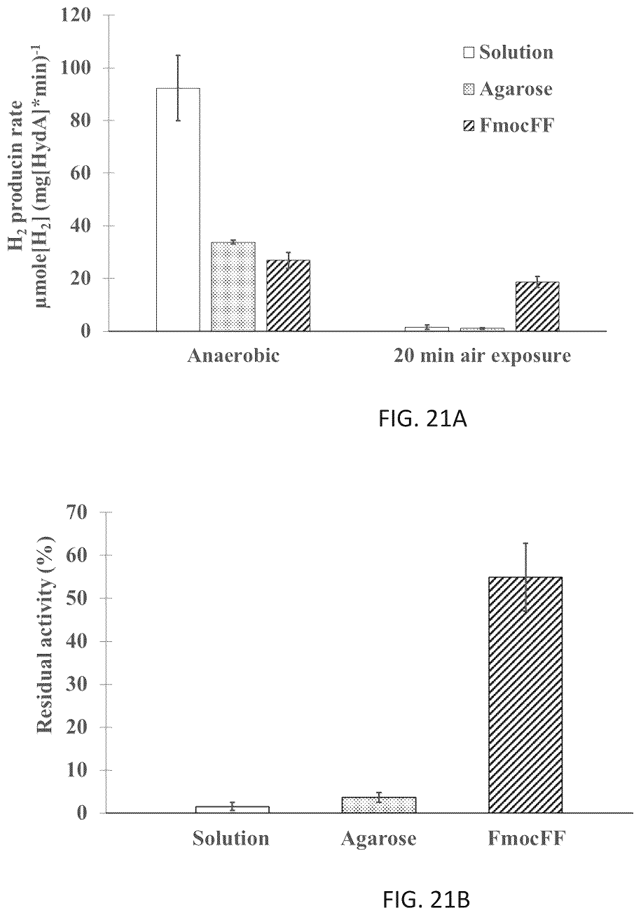

As shown in FIGS. 21A and 21B, HydA enzymatic H₂ production in exemplary FmocFF hydrogel was more than 1.5 orders of magnitude greater than in agarose hydrogels or in solution.

These results indicate that exemplary aromatic peptide-based hydrogels provide a surprising degree of protection against oxygen.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-47. (canceled)

48. A method of controlling a concentration of free oxygen in an environment, the method comprising contacting said environment with a self-assembled structure formed of a plurality of peptides each being of 2 to 6 amino acid residues in length, wherein in each of said peptides, at least one of said amino acid residues is an aromatic amino acid residue, and wherein said self-assembled structure is capable of interacting with oxygen, thereby controlling the concentration of free oxygen, wherein said self-assembled structure is in a form of a hydrogel.

49. The method of claim 48, comprising contacting said environment with a plurality of discrete hydrogel particles each comprising said self-assembled structure.

50. The method of claim 48, wherein at least one, or each, peptide in said plurality of peptides comprises an end-capping moiety.

51. The method of claim 50, wherein said end-capping moiety is aromatic and is attached to the N-terminus of said peptide.

52. The method of claim 48, wherein said peptide is a dipeptide.

53. The method of claim 52, wherein said dipeptide comprises diphenylalanine.

54. The method of claim 53, wherein said dipeptide is Fmoc-diphenylalanine.

55. The method of claim 48, wherein said self-assembled structure is capable of absorbing oxygen in an amount of at least 0.02 mg oxygen per mg of said self-assembled structure.

56. The method of claim 48, wherein:
said environment is a fluid environment; and/or
said environment is or comprises an animate substrate.

57. The method of claim 48, wherein controlling the concentration of free oxygen is reducing a concentration of the free oxygen in the environment.

58. The method of claim 48, wherein controlling the concentration of free oxygen is in an environment external and/or internal to the self-assembled structure.

59. The method of claim 48, wherein the self-assembled structure further comprises an oxygen-sensitive substance incorporated therein, the method being for protecting or maintaining an activity of the oxygen-sensitive substance; and/or wherein the self-assembled structure further comprises a substance which participates in an oxygen-sensitive reaction incorporated therein, the method being for performing said reaction under aerobic conditions.

60. An article-of-manufacturing comprising a self-assembled structure as defined in claim 48 and an oxygen-sensitive substance.

61. A method of transporting oxygen from a first environment to a second environment, the method comprising:
contacting said first environment with a self-assembled structure as defined in claim 48 to obtain said self-assembled structure having oxygen interacted therewith; and
contacting said second environment with said self-assembled structure having oxygen interacted therewith, thereby releasing oxygen from said self-assembled structure into said second environment.

62. The method of claim 61, wherein said second environment comprises a tissue, the method being for enhancing healing of tissue damage.

63. The method of claim 61, being for:
inhibiting growth of an anaerobic organism in said second environment; and/or
enhancing aerobic metabolism in said second environment.

64. A self-assembled structure as defined in claim 48 having oxygen interacted therewith.

65. A composition comprising a substance which is oxygen-sensitive and/or which participates in an oxygen-sensitive reaction, said substance being incorporated in a self-assembled structure as defined in claim 48.

66. The composition of claim 65, wherein said substance is an enzyme.

67. A system comprising the composition of claim 65, and a current collector configured for being attached to an electrical power source.

68. A method of reducing or oxidizing a substrate, the method comprising contacting said substrate with the composition of claim 65, and applying electrical power to said composition, thereby reducing or oxidizing the substrate.

69. The method of claim 68, wherein said substance comprises a hydrogenase, the method being for producing H₂.

70. The method of claim 68, wherein said substance comprises a nitrogenase, the method being for producing ammonia and/or a salt thereof.

71. An article-of-manufacturing comprising a substance which is oxygen-sensitive incorporated in a self-assembled structure as defined in claim 48.
